(19) United States
(12) Patent Application Publication
(10) Pub. No.: US 2026/0335277 A1
(43) Pub. Date: Oct. 29, 2026

(54) CHLOROTONIL DERIVATIVES

(71) Applicant: HELMHOLTZ-ZENTRUM FUR INFEKTIONSFORSCHUNG GMBH, Braunschweig (DE)

(72) Inventors: Walter Hofer; Felix Deschner; Jennifer Herrmann, Braunschweig (DE); Rott Müller, Braunschweig (DE); Marc Stadler; Mriram Grosse; Jana Held; Lais PessanhabDebCarvalho; Markus Bischoff; Linda Pätzold

(21) Appl. No.: 19/483,097

(22) PCT Filed: May 8, 2024
(86) PCT No.: PCT/EP2024/062793

§ 371 Date: Nov. 10, 2025

(30) Foreign Application Priority Data
May 10, 2023 (EP) 23172607.6

(51) Int. Cl. A61K 31/365; A61P 31/04; C07D 313/00; C07D 493/04

(52) U.S. Cl. CPC A61K 31/365; A61P 31/04; C07D 313/00; C07D 493/04

(57) ABSTRACT

The present invention relates to novel chlorotonil derivatives of formula (I) and the use thereof for the treatment or prophylaxis of bacterial infections and malaria.

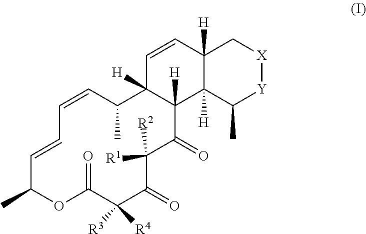

The present invention provides novel chlorotonil derivatives and the use thereof for the treatment or prophylaxis of bacterial infections and malaria.

The present invention relates to the field of infectious diseases caused by bacterial pathogens and plasmodia parasites and the need of novel antibiotics for therapeutic applications. The compounds of the present invention act against often multi-drug resistant Gram-positive bacteria such as Staphylococcus spp, and Enterococcus spp., M. tuberculosis, and the parasite Plasmodium falciparum addressing a novel antibacterial target. The present invention inter alia tackles the development of a new antimalarial agent with a fast clearance rate of asexual parasites and additional sexual blood stage activity against Plasmodium falciparum with a presumed novel mode-of-action.

Chlorotonil A is a highly lipohilic tricyclic macrolide consisting of an unusual gem-dichloro-1,3-dione moiety, a novel feature among natural polyketides (Gerth K, Steinmetz H, Höfle G, Jansen R. Chlorotonil A, a macrolide with a unique gem-dichloro-1,3-dione functionality from Sorangium cellulosum, So ce1525. Angew Chem Int Ed Engl 2008, 47 (3), 600-602). It is produced by the myxobacterium Sorangium cellulosum, a Gram-negative soil-dwelling bacterium. It showed very promising antiplasmodial activity in the low nanomolar range in a first screening, testing for in vitro activity against chloroquine-sensitive and chloroquine-resistant laboratory strains of Plasmodium falciparum (mean IC₅₀: 9 and 18 nM, respectively) and against clinical isolates from Gabon (median IC₅₀: 15.2 nM) (Jungmann K, Jansen R, Gerth K, Huch V, Krug D, Fenical W, Müller R. Two of a Kind—The Biosynthetic Pathways of Chlorotonil and Anthracimycin. ACS Chem Biol 2015, 10 (11), 2480-2490; Held J, Gebru T, Kalesse M, Jansen R, Gerth K, Müller R, Mordmüller B. Antimalarial activity of the myxobacterial macrolide chlorotonil A. Antimicrob Agents Chemother 2014, 58 (11), 6378-6784). It showed a very rapid onset of action in vitro; already after 1 h exposure and subsequent washing of the culture, the IC₅₀ was only 1.3-fold higher compared to the IC₅₀ when no washing step was applied. When tested for the inoculum effect, chlorotonil A showed nearly no variation in the IC₅₀ (1.2 fold difference between lowest and highest parasitemia) when assays were performed at starting parasitemia levels of 0.01, 0.05, 0.5 and 2.5%. Chloroquine and artesunate on the other hand showed a higher IC₅₀ when higher parasitemia levels were used (5.6- and 8.8-fold, respectively). So far, it was not possible to develop resistant parasites in vitro by drug pressure. Chlorotonil A showed very promising gametocytocidal activity against mature stage (stage IV-V) gametocytes in a bioluminescence assay measuring ATP. The obtained mean IC₅₀ was 9.5 nM displaying a superior activity to all other tested compounds in this assay, besides epoxomicin, which shows toxic effects in vivo. First in vivo experiments in mice showed that chlorotonil A is orally available and that it displays in vivo efficacy (4 day suppression test; activity >90% reduced parasitemia at different doses of 36-110 mg/kg in a pilot experiments). However, correct dosing in the mouse model was very difficult. The compound could only be given in solid form together with peanut butter, which hampers the ability to propose a correct and accurate dosing scheme.

Another natural derivative is chlorotonil B1, resulting from the loss of one of the chlorine atoms with a keto-enol group. The tautomer B2, the mixed sodium/magnesium salt, is obtained after silica gel purification of chlorotonil B1 or by heating ChB1 in toluene with the respective carbonates.

The maximum tolerated concentration (MTC) of chlorotonil A and B through preliminary toxicity studies in zebrafish could not be determined because of observed precipitation in stock solutions and assay wells. Nonetheless, the MTC of new derivatives (ChA-Epo2 and ChB-Epo) was determined, and showed that the toxicological profile can be optimized using semisynthetic derivatisation.

In a subsequent study, chlorotonil A and chlorotonil B1 were epoxidized at C9/10 and the epoxide was opened (W. Hofer, E. Queis, A. A. Fayad, F. Deschner, A. Andreas, L. P. de Carvalho, S. Hüttel, S. Bernecker, L. Pätzold, B. Morgenstern, N. Zaburannyi, M. Bischoff, M. Stadler, J. Held, J. Herrmann, R. Müller, Angew. Chem. Int. Ed. 2022, e202202816; WO 2019/092030 A1). All derivatives were better soluble whereas activity in Gram-positive bacteria was retained in most cases. Nevertheless, efficacy against malaria dropped significantly in vitro and in vivo.

Chlorotonil A shows poor water solubility which limits the application and further development. In addition, standard in vitro ADME parameters cannot be uniquely determined since chlorotonil A concentrations in these assays were always below the limit of detection. The same holds true for the partly improved semi-synthetic epoxidized chlorotonil B1 (B1-Epo2) that showed increased solubility as well as bioavailability but lacked efficacy in P. falciparum infection models (50 mg/kg chlorotonil A per oral: full cure; 100 mg/kg chlorotonil B1-Epo2 per oral: 7-fold reduction of parasitemia compared to placebo and no cure). Both compounds (chlorotonil A and B1-Epo2) could be administered at low doses intravenously, however, only when dissolved in a vehicle which is not suitable for further development as part of preclinic studies as it contained high amounts of organic solvents (10 vol. % ethanol, 5 vol. % DMSO). In addition to these general drawbacks, toxicity was observed for chlorotonil A in mouse models after multiple PO dosing at 100 mg/kg (general adverse effects, and cardio- and hepatotoxicity). In comparative studies using zebrafish larvae models both chlorotonil A and B1-Epo2 displayed cardiotoxic, hepatotoxic and developmental toxicity effects.

Therefore, new derivatives are needed to overcome problems that are associated with solubility, bioavailability, stability, and efficacy in all species. Accordingly, it was an object of the present invention to provide new derivatives of the known chlorotonils which overcome the drawbacks of the state of the art.

The present invention provides compounds of general formula (I):

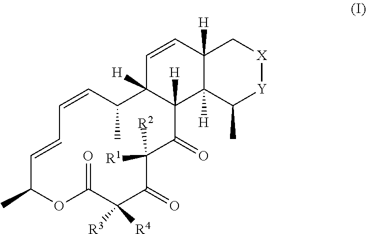

wherein
X—Y together are a group of formula

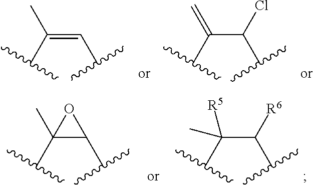

R¹ is hydrogen, F, Cl, S₂H or a methyl group;
R² is hydrogen, F, Cl or a methyl group;
R³ is hydrogen or a methyl group;
R⁴ is hydrogen or a methyl group; and
R⁵ is OH and R⁶ is a halogen atom, OH, ONO₂ or a group of formula —O—C₁₋₆ alkyl which group may be substituted by one or two hydroxy groups and/or by a phenyl group; or
R⁵ is a halogen atom, OH, ONO₂ or a group of formula —O—C₁₋₆ alkyl which group may be substituted by one or two hydroxy groups and/or by a phenyl group and R⁶ is OH;
or a salt thereof.

The following compounds are excluded from the present invention:

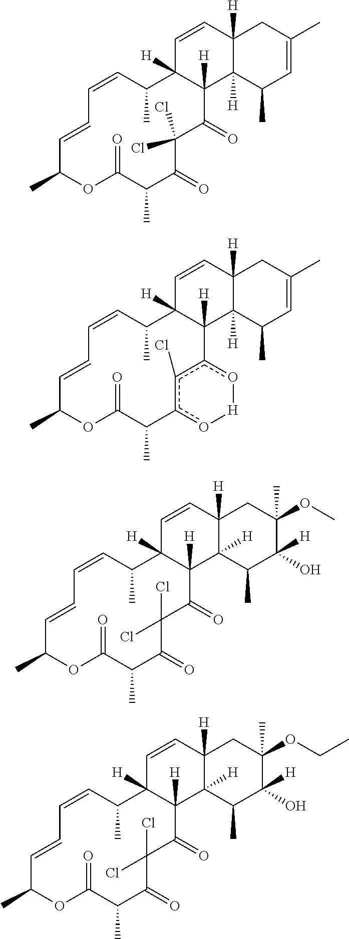

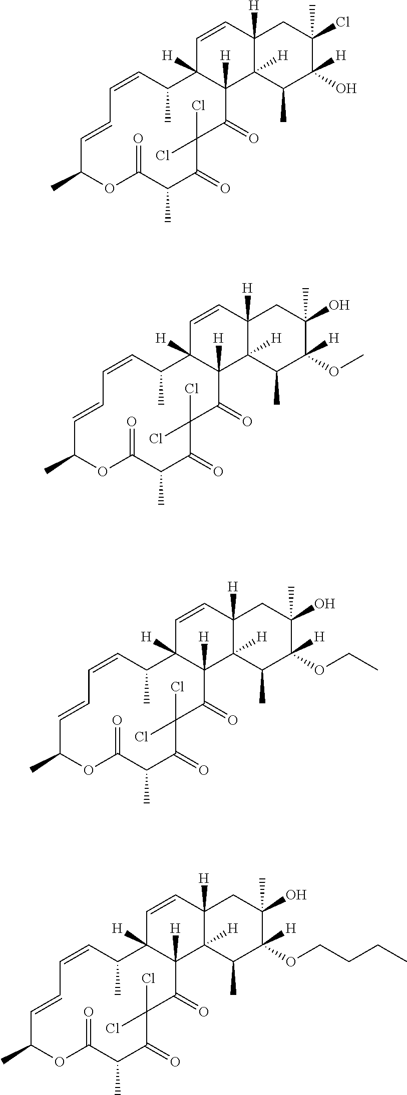

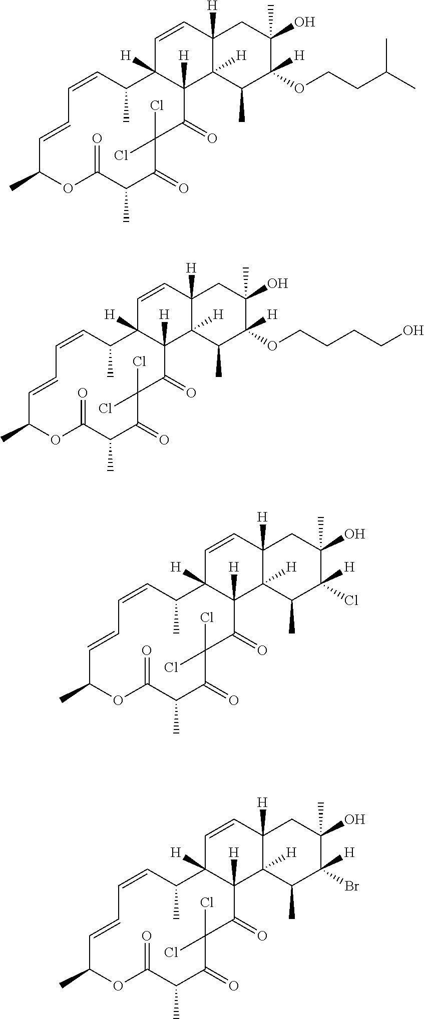

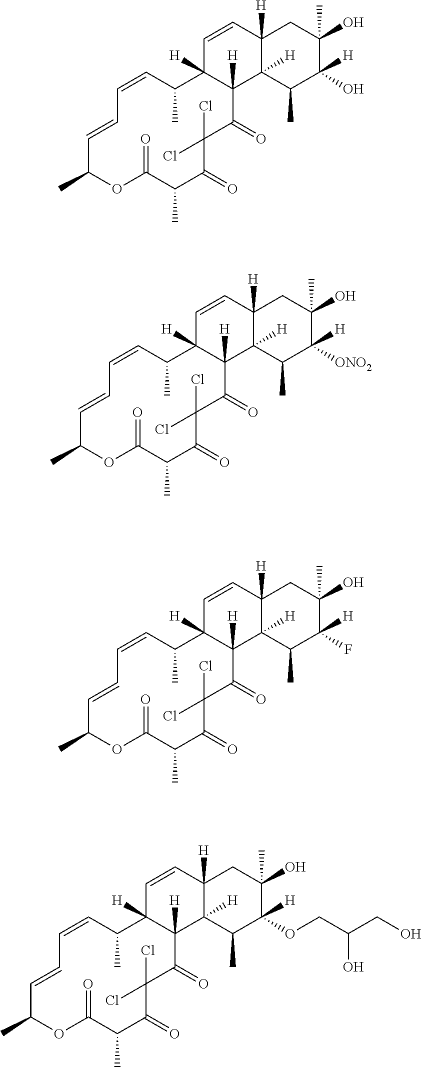

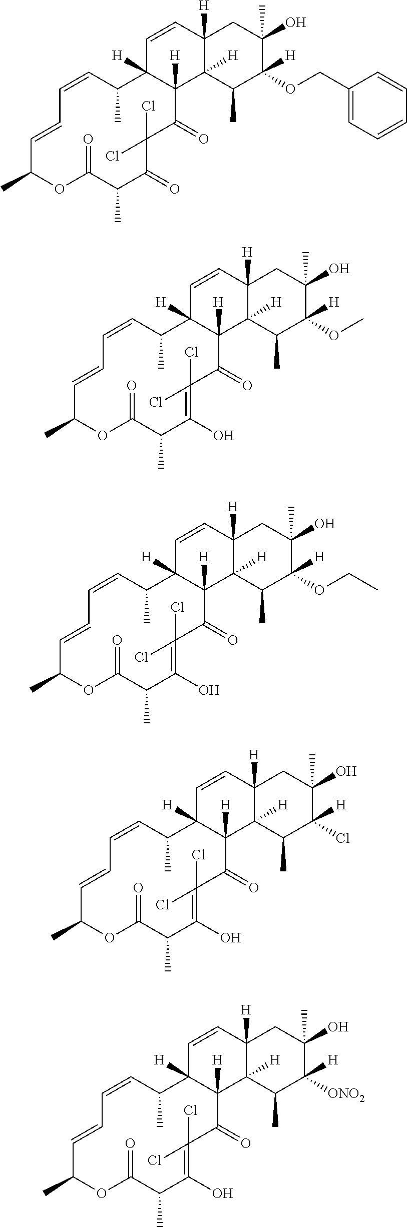

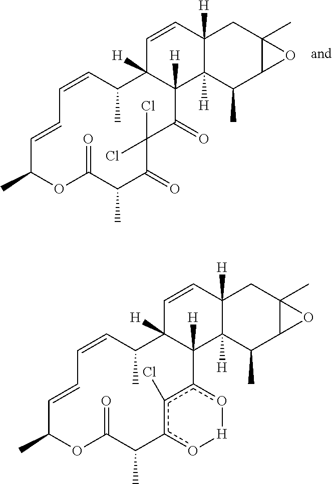

and all tautomeric forms and all salts thereof.

The compound disclosed in WO 2019/092030 and all tautomeric forms and all salts thereof are excluded from the present invention.

According to a preferred embodiment, the following compounds and all tautomeric forms and all salts thereof as such are excluded from the present invention:

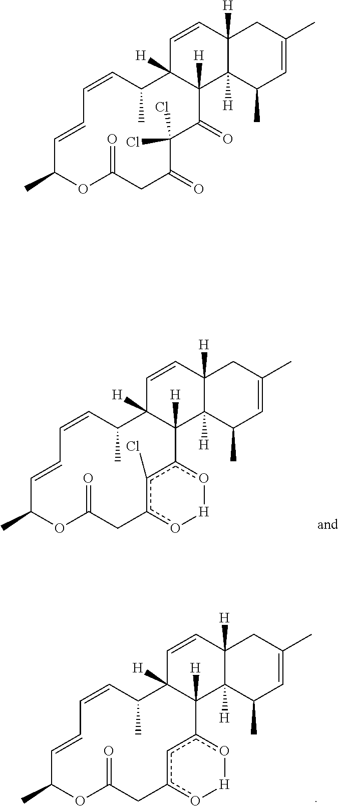

According to a further preferred embodiment, the use of these compounds in the treatment or prophylaxis of bacterial infections (especially the use thereof in the treatment or prophylaxis of a bacterial infection caused by Staphylococcus spp, and/or Enterococcus spp. (e.g., by S. aureus)) is encompassed by the present invention.

According to a moreover preferred embodiment, the use of these compounds in the treatment or prophylaxis of malaria is encompassed by the present invention.

According to a further preferred embodiment, the compounds and all tautomeric forms and all salts thereof disclosed in Jungmann K, Jansen R, Gerth K, Huch V, Krug D, Fenical W, Müller R. Two of a Kind—The Biosynthetic Pathways of Chlorotonil and Anthracimycin. ACS Chem Biol 2015, 10 (11), 2480-2490 (and the supporting information) as such are excluded from the present invention.

According to a preferred embodiment, the present invention provides compounds of general formula (II):

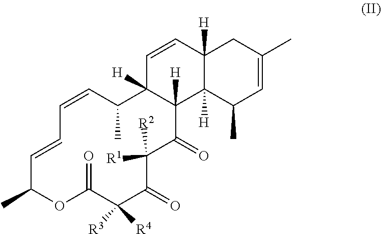

wherein
R¹ is hydrogen, F, Cl, S₂H or a methyl group;
R² is hydrogen, F, Cl or a methyl group;
R³ is hydrogen or a methyl group; and
R⁴ is hydrogen or a methyl group;
or a salt thereof.

According to a further preferred embodiment, the present invention provides compounds of general formula (III):

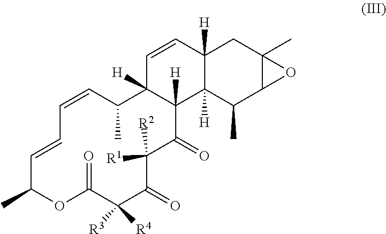

wherein
R¹ is hydrogen, F, Cl, S₂H or a methyl group;
R² is hydrogen, F, Cl or a methyl group;
R³ is hydrogen or a methyl group; and
R⁴ is hydrogen or a methyl group;
or a salt thereof.

Preferably, R¹ is hydrogen or Cl.

Further preferably, R¹ is hydrogen.

Moreover preferably, R² is F or a methyl group.

Further preferably, R² is hydrogen.

Moreover preferably, R³ is a methyl group.

Further preferably, R⁴ is hydrogen.

According to a further preferred embodiment, the present invention provides compounds of general formula (IV):

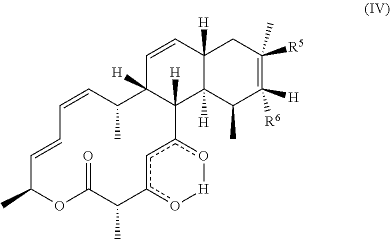

wherein
R⁵ is OH and R⁶ is a halogen atom, OH, ONO₂ or a group of formula —O—C₁₋₆ alkyl which group may be substituted by one or two hydroxy groups and/or by a phenyl group; or
R⁵ is a halogen atom, OH, ONO₂ or a group of formula —O—C₁₋₆ alkyl which group may be substituted by one or two hydroxy groups and/or by a phenyl group and R⁶ is OH;
or a salt thereof.

Further preferably, R⁵ is Cl, OH, ONO₂, OMe, OEt, OnBu, Oisoamyl, OBenzyl or glycerol; and R⁶ is OH.

Moreover preferably, R⁶ is Cl, OH, ONO₂, OMe, OEt, OnBu, Oisoamyl, OBenzyl or glycerol; and R⁵ is OH.

Compounds of the present invention, wherein X—Y together are a group of formula

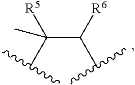

can be prepared as described in WO 2019/092030.

Preferably, the compound of the present invention is the following compound (“dehalogenil”), or a salt thereof:

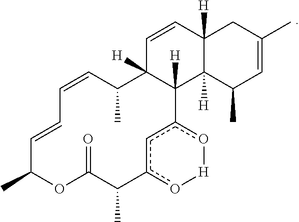

Moreover preferably, the compound of the present invention is the following compound, or a salt thereof:

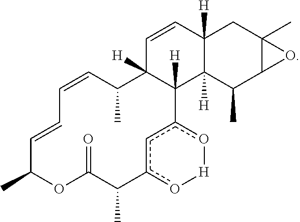

Further preferably, the compound of the present invention is selected from the following compounds, or a salt thereof:

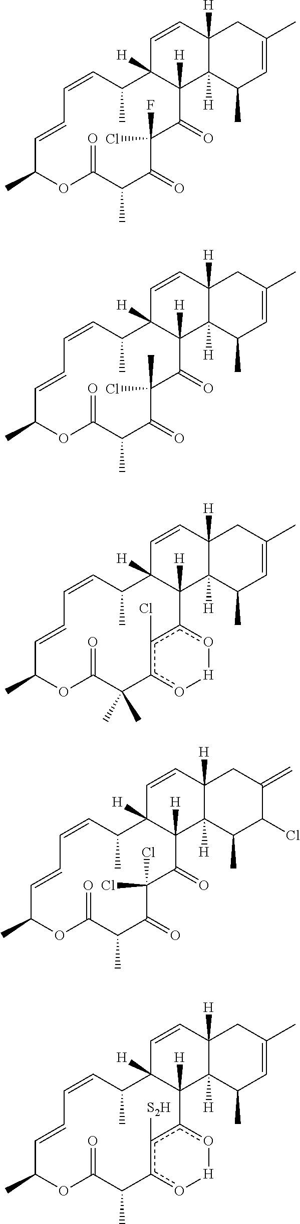

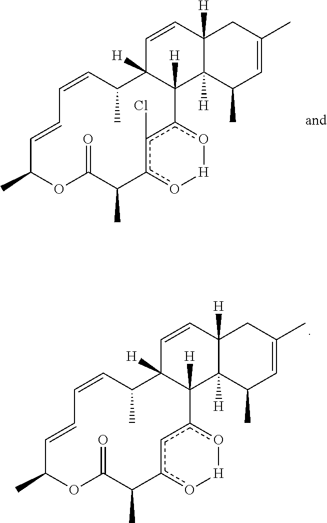

Especially preferred are the compounds disclosed in the examples or a salt thereof.

It is further preferred to combine the preferred embodiments of the present invention in any desired manner (e.g., any embodiment for R¹ may be combined with any embodiment of R⁴).

The present invention further provides a method for the preparation of the following compound:

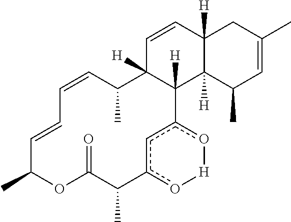

which is characterized by that chlorotonil B1:

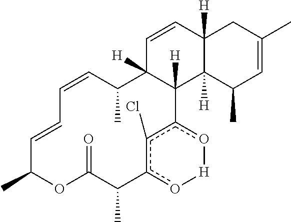

is reacted with sulfur and KOtBu (preferably in dimethylformamide, DMF).

The present invention moreover provides a method for the preparation of the following compound:

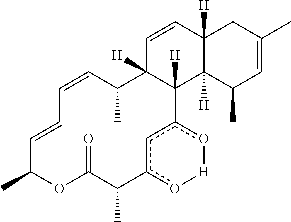

which is characterized by that chlorotonil A:

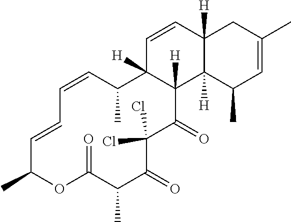

is irradiated by blue LEDs in the presence of (Ir[dF(CF₃)ppy]₂(dtbpy))PF₆, formic acid and DIPEA.

The expression C₁₋₆ alkyl refers to a saturated, straight-chain or branched hydrocarbon group that contains from 1 to 6 carbon atoms. Examples are a methyl (Me), ethyl, propyl, iso-propyl, n-butyl, iso-butyl, sec-butyl or a tert-butyl group.

The present invention includes all tautomeric forms of the compounds of the present invention. The present invention especially includes all tautomeric forms of compounds of formula (I) or (II), wherein R¹ and/or R² are hydrogen. For example, the compound dehalogenil includes the following tautomeric forms:

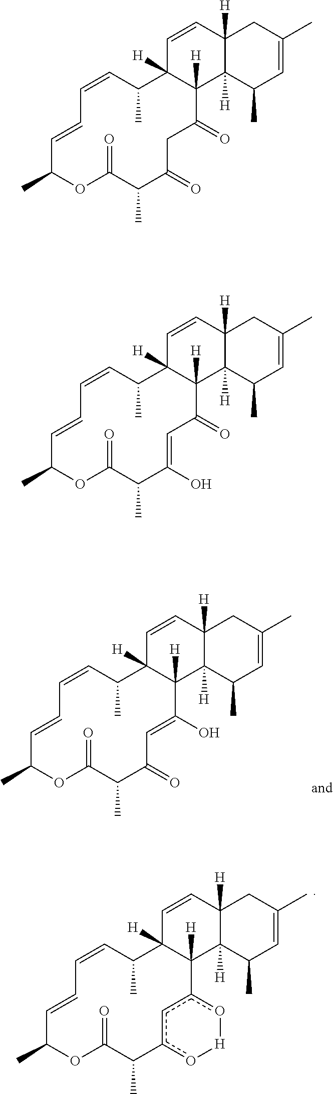

Owing to the substitution at group X—Y, the compounds of the present invention may contain one or more centres of chirality at this position. Here, the present invention includes all pure enantiomers as well as all pure diastereomers and also mixtures thereof in any mixing ratio.

The present invention further provides pharmaceutical compositions comprising one or more compounds described herein or a salt thereof, optionally in combination with one or more carrier substances and/or one or more adjuvants.

The present invention furthermore provides compounds or pharmaceutical compositions as described herein for use in the treatment and/or prophylaxis of bacterial infections; especially for the treatment and/or prophylaxis of a bacterial infection caused by Staphylococcus spp. and/or Enterococcus spp. (e.g., by S. aureus).

The present invention furthermore provides compounds or pharmaceutical compositions as described herein for use in the treatment and/or prophylaxis of malaria.

It is a further object of the present invention to provide a compound as described herein or a pharmaceutical composition as defined herein for the preparation of a medicament for the treatment and/or prophylaxis of bacterial infections; especially for the treatment and/or prophylaxis of a bacterial infection caused by Staphylococcus spp, and/or Enterococcus spp. (e.g., by S. aureus).

It is a further object of the present invention to provide a compound as described herein or a pharmaceutical composition as defined herein for the preparation of a medicament for the treatment and/or prophylaxis of malaria.

The present invention further provides a method for the treatment or prophylaxis of malaria or of a bacterial infection (especially a bacterial infection caused by Staphylococcus spp, and/or Enterococcus spp. (e.g., by S. aureus)) in a subject which comprises administering to the subject an effective amount of a compound of the present invention.

The compounds of the present invention are especially useful in the treatment and prophylaxis of infections caused by Gram-positive bacteria such as Staphylococcus spp., Enterococcus spp., M. tuberculosis, Clostridium spp, and the parasite Plasmodium falciparum.

Examples of salts (especially of pharmacologically acceptable salts) of sufficiently acidic compounds are alkali or earth alkali metal salts, for example sodium, potassium, lithium, calcium or magnesium salts; ammonium salts; or organic base salts, for example methylamine, dimethylamine, ethylenediamine, ethanolamine, choline hydroxide, meglumin, piperidine, morpholine, tris-(2-hydroxyethyl)amine, lysine or arginine salts.

The compounds described herein may be solvated, especially hydrated. The hydratization/hydration may occur during the process of production or as a consequence of the hygroscopic nature of the initially water free compounds. The solvates and/or hydrates may e.g. be present in solid or liquid form.

The therapeutic use of the compounds described herein, their salts, solvates and hydrates, respectively, as well as formulations and pharmaceutical compositions also lie within the scope of the present invention.

As mentioned above, therapeutically useful agents that contain compounds described herein, their solvates, hydrates, salts or formulations are also comprised in the scope of the present invention. In general, the compounds described herein will be administered by using the known and acceptable modes known in the art, either alone or in combination with any other therapeutic agent.

For administration, such therapeutically useful agents can be administered by one of the following routes: oral, e.g. as tablets, dragees, coated tablets, pills, semisolids, soft or hard capsules, for example soft and hard gelatine capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatine, capsules the therapeutically useful product may be mixed with pharmaceutically inert, inorganic or organic excipients as are e.g. lactose, sucrose, glucose, gelatine, malt, silica gel, starch or derivatives thereof, talc, stearinic acid or their salts, dried skim milk, and the like. For the production of soft capsules one may use excipients as are e.g. vegetable, petroleum, animal or synthetic oils, wax, fat, and polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Especially preferred are lipids and more preferred are phospholipids (preferred of natural origin; especially preferred with a particle size between 300 to 350 nm) preferred in phosphate buffered saline (pH=7 to 8, preferred 7.4). For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilization, e.g. UV stabilizers, emulsifiers, sweetener, aromatizers, salts to change the osmotic pressure, buffers, coating additives and antioxidants.

In general, in the case of oral or parenteral administration to adult humans weighing approximately 80 kg, a daily dosage of about 1 mg to about 10,000 mg, preferably from about 5 mg to about 1,000 mg, should be appropriate, although the upper limit may be exceeded when indicated. The daily dosage can be administered as a single dose or in divided doses, or for parenteral administration, it may be given as continuous infusion or subcutaneous injection.

The novel chlorotonil derivatives of the present invention are significantly improved compared to chlorotonil A and chlorotonil B1-Epo2 in terms of their pharmacological properties. Furthermore, different substitution patterns at different moieties show selectivity against different strains, omitting potential resistance development in other pathogens.

The compounds of the present invention (especially the compound “dehalogenil”) show significantly improved properties allowing various routes of administration (e.g., PO, IV, SC, IP) in rodent models and superior in vivo activity was demonstrated in mouse models of S. aureus infection (sepsis and catheter-induced infection) following SC administration. Other issues with the known compounds (in particular chlorotonil A and chlorotonil B1-Epo2) were their high lipophilicity and therefore low aqueous solubility, partly insufficient in vivo potency (ChB1-Epo2), and adverse effects in mice (ChA) at higher doses. The compounds of the present invention address these issues. In particular, dehalogenil showed further increased solubility, bioavailability and stability, and a more favourable safety margin when compared to former tested chlorotonil derivatives.

The new derivatives of the present invention address different parts of the molecule revealing important moieties for activity and selectivity. For example, dehalogenil, exhibits doubled aqueous solubility when compared to chlorotonil B1-Epo2 being already superior to chlorotonil A. Furthermore, activity similar to chlorotonil A was maintained parallel to a significantly improved safety profile in zebrafish larvae and rodents. Because of its higher solubility, dehalogenil can be formulated to be injected. 3-fold higher plasma levels could be detected and an oral bioavailability of ca. 10% could be determined. Furthermore, dehalogenil reduced the P. berghei parasite load in mice by >99%. In a S. aureus sepsis model, subcutaneous administration of dehalogenil (10 and 25 mg/kg) increased survival rates comparable to a higher dose of linezolid (60 mg/kg, standard care). Similarly, dehalogenil (6×25 mg/kg, SC) was highly effective in a mouse model of catheter-induced infection by significantly reducing bacterial loads at the site of infection and on the implant. In zebrafish models, no toxic effects were observed at 5-10 μg/mL dehalogenil, whereas ChA and ChB1-Epo2 showed toxic signs already below 1 pg/mL.

The structure of the compound dehalogenil has been mentioned in the literature (N. Rahn, M. Kalesse, Angew. Chem. Int. Ed. 2008, 47, 597-599; Dissertation Nicola Rahn [https://d-nb.info/985296003/34]; Dissertation Anastasie Kena Diba [https://www.repo.uni-hannover.de/bitstream/handle/123456789/7487/626773628.pdf?sequence=1]; Jana Held, Tamirat Gebru, Markus Kalesse, Rolf Jansen, Klaus Gerth, Rolf Müller, Benjamin Mordmüller, Antimicrobial Agents and Chemotherapy 2014 Vol. 58, No. 11, 6378-6384). According to these publications, dehalogenil was made by total synthesis, or by dehalogenation of chlorotonil A by either SmI₂ or ascorbic acid/triethylamine.

However, when the published NMR spectra were compared with the NMR spectra of dehalogenil of the present invention, it became apparent that the proposed molecule in the literature is in fact not dehalogenil. Further, in the literature, dehalogenil is described as inactive, whereas it has been found that dehalogenil derived from semi-synthesis according to the present invention (dehalogenation of chlorotonil B1 by sulfur/KOtBu) is similar active as chlorotonil A without its drawbacks. The crystal structure of dehalogenil that is presented herein clearly confirms that the molecule that has been synthesized according to the present invention is in fact dehalogenil.

### EXAMPLES

### Analysis of Dehalogenil Described in the Literature:

As mentioned above, the preparation of the chlorine free chlorotonil derivative (dehalogenil) was described previously in the literature by three methods:
1) total synthesis^{1,2}
2) dehalogenation of chlorotonil A (ChA) by ascorbic acid/triethylamine³
3) dehalogenation of ChA by SmI₂²

### LITERATURE

[1] N. Rahn, M. Kalesse, Angew. Chem. Int. Ed. 2008, 47, 597-599 and Supporting Information
[2] Dissertation Nicola Rahn, Gottfried Wilhelm Leibniz Universität Hannover, 2007
[3] Dissertation Anastasie Kena Diba, Gottfried Wilhelm Leibniz Universität Hannover, 2010

The inventors of the present invention developed a new method to dehalogenate the naturally occurring chlorotonil B1 (ChB1) to dehalogenil.

In the following, it will be explained why the formerly used methods led to derivative(s) other than dehalogenil. Spectra and reactions mentioned can be found in the attached figures.

First the HPLC/MS spectra and the most prominent peaks in the NMR are described and later the products of the different, former methods to obtain dehalogenil are compared.

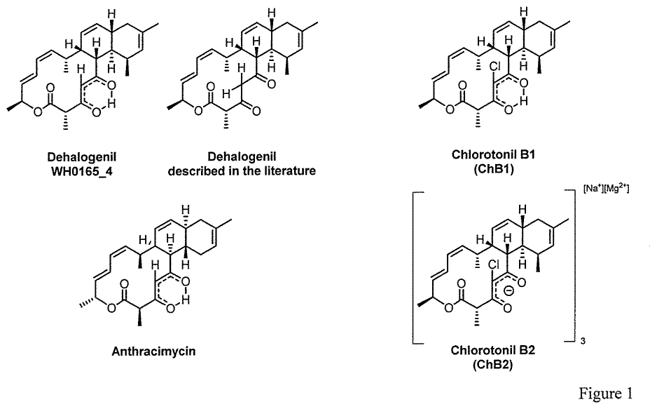

The structures of dehalogenil, the structure of dehalogenil proposed in the literature and the structures of the naturally occurring isomer anthracimycin, chlorotonil B1 and chlorotonil B2 are shown in FIG. 1.

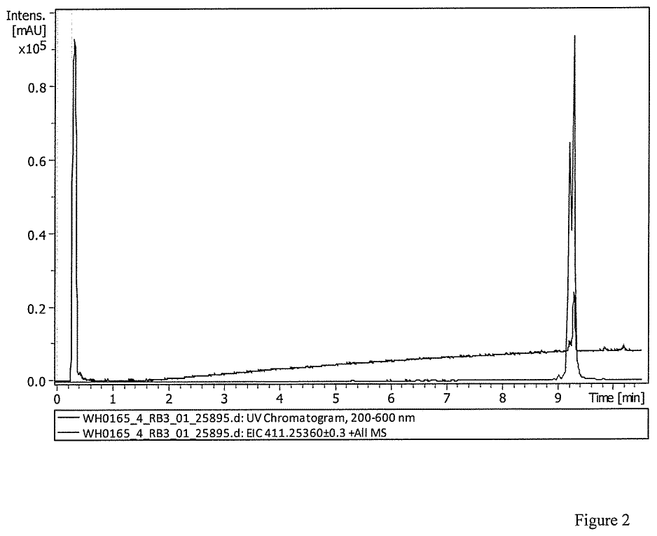

FIG. 2 shows the HPLC/MS spectra of WH0165_4/Dehalogenil.

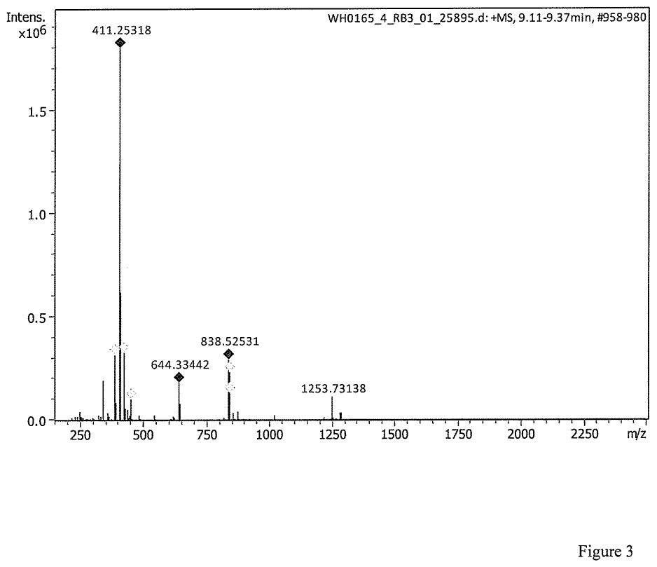

FIG. 3 shows the MS spectrum of the largest UV peak of WH0165_4/Dehalogenil.

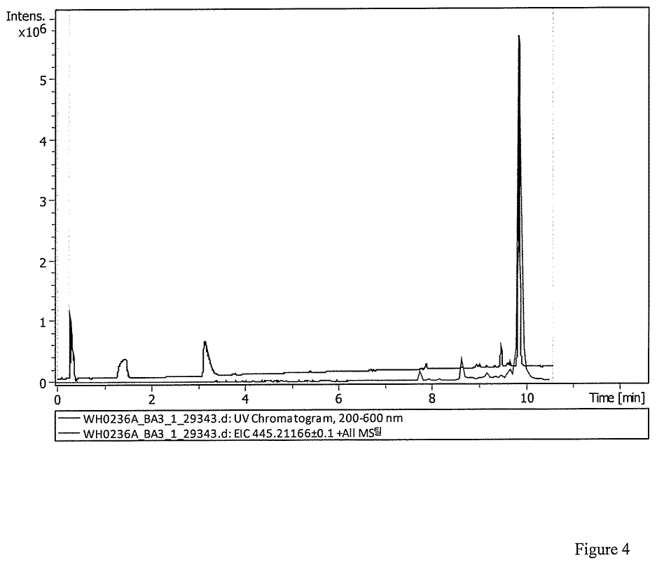

FIG. 4 shows the HPLC/MS spectra of the product of the dehalogenation of ChA with ascorbic acid/TEA.

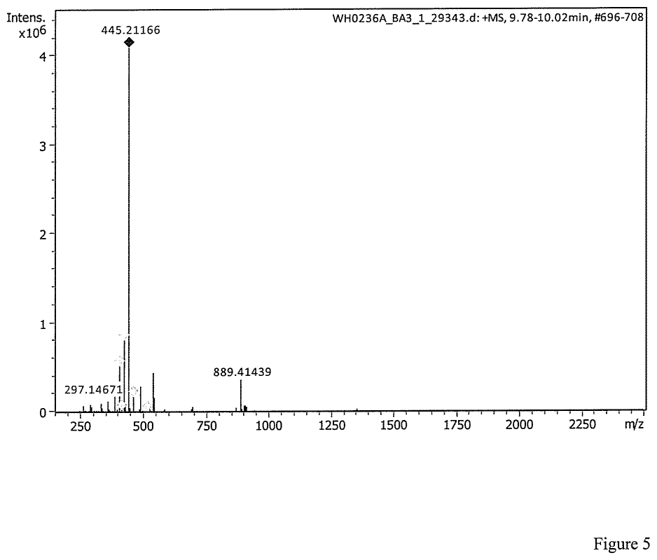

FIG. 5 shows the MS spectrum of the largest UV peak of the product of the dehalogenation of ChA with ascorbic acid/TEA.

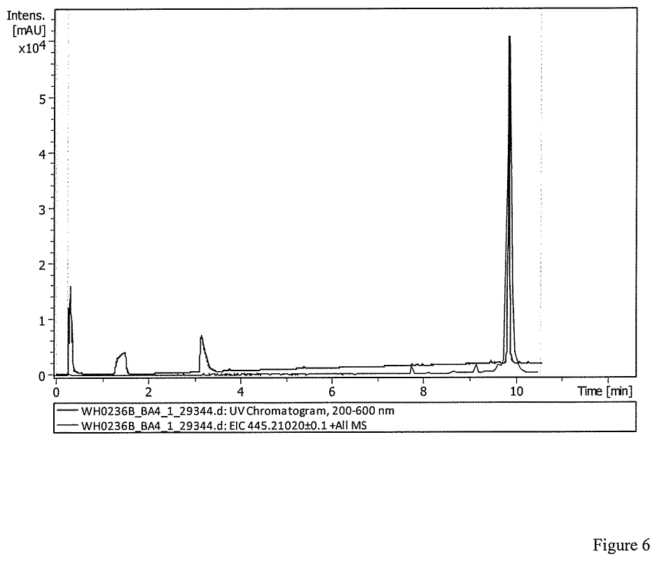

FIG. 6 shows the HPLC/MS spectra of the product of the dehalogenation of ChA with SmI₂.

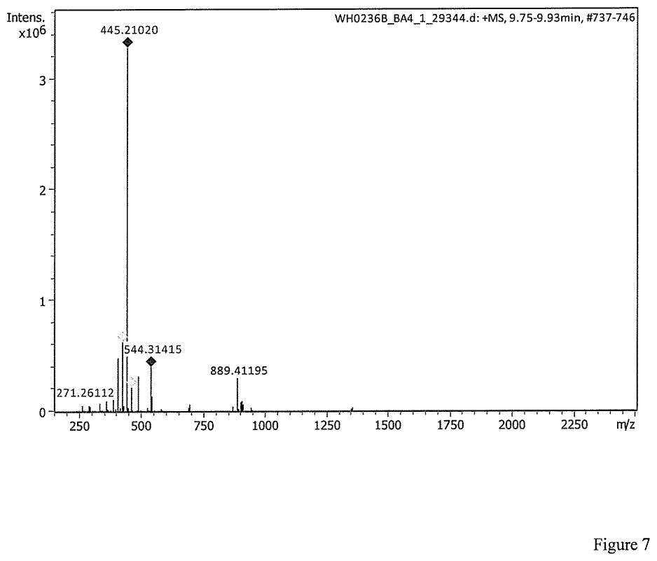

FIG. 7 shows the MS spectrum of the largest UV peak of the product of the dehalogenation of ChA with SmI₂.

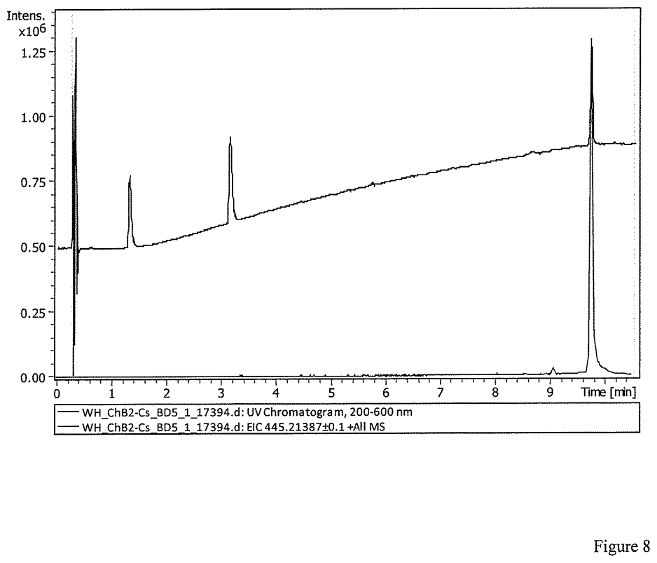

FIG. 8 shows the HPLC/MS spectra of ChB1/ChB2.

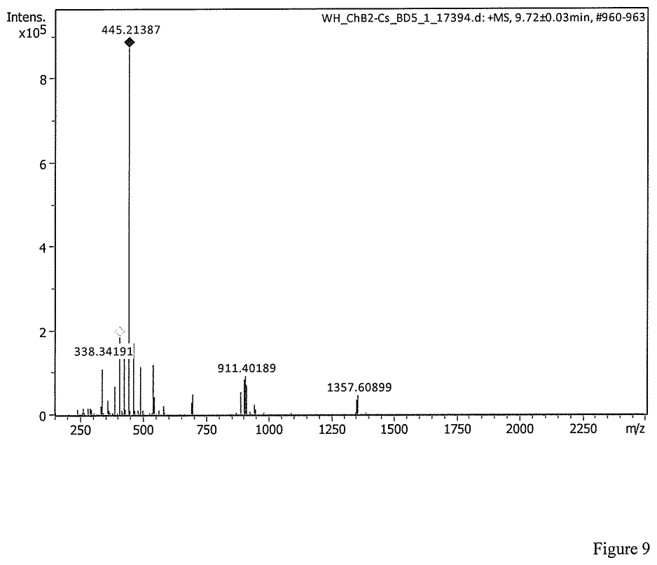

FIG. 9 shows the MS spectrum of the largest UV peak of ChB1/ChB2.

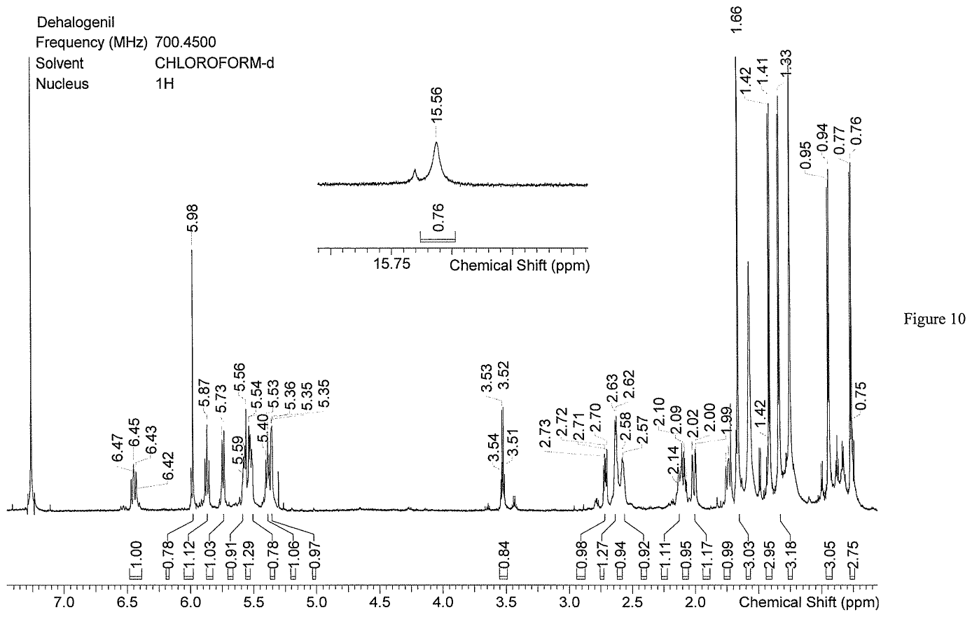

FIG. 10 shows the ¹H-NMR spectra of dehalogenil of the present invention (WH0165_4).

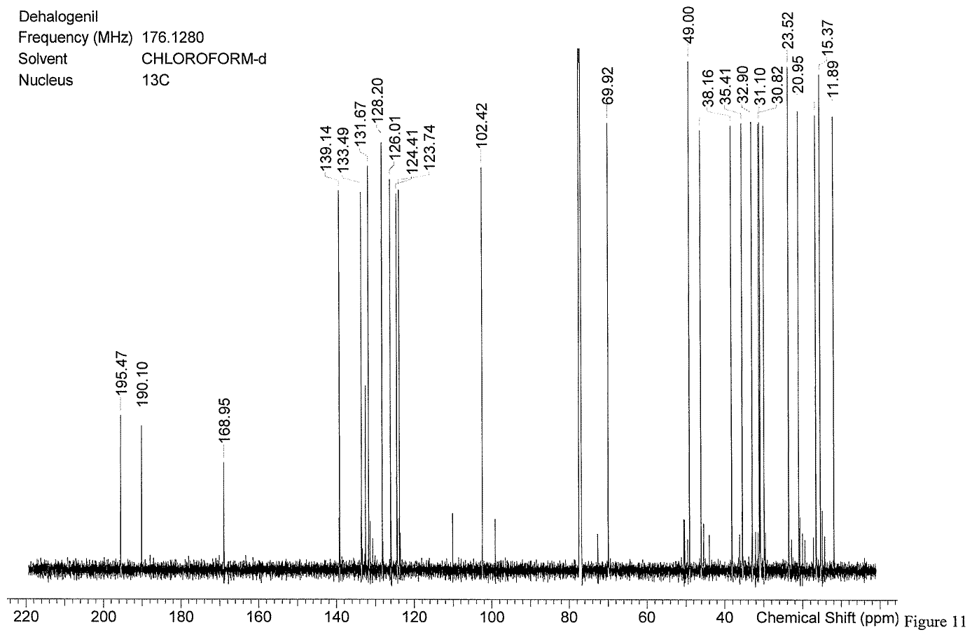

FIG. 11 shows the ¹³C-NMR spectra of dehalogenil of the present invention (WH0165_4).

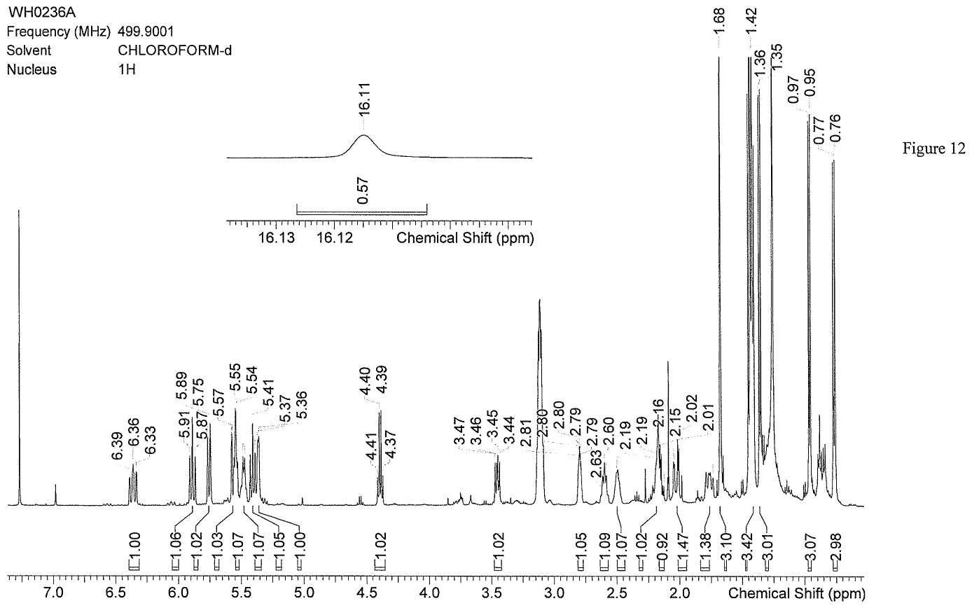

FIG. 12 shows the ¹H-NMR spectra of WH0236A.

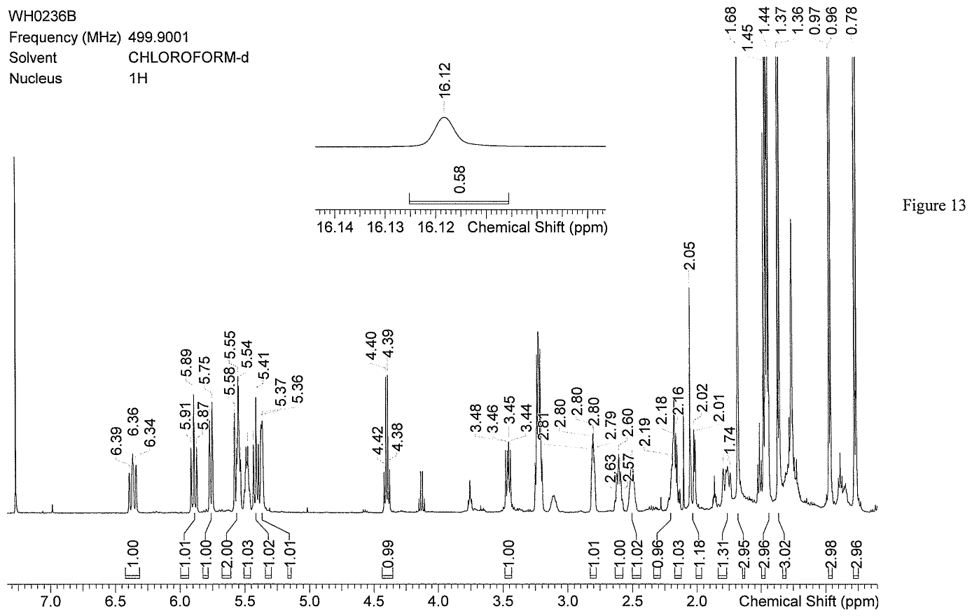

FIG. 13 shows the ¹H-NMR spectra of WH0236B.

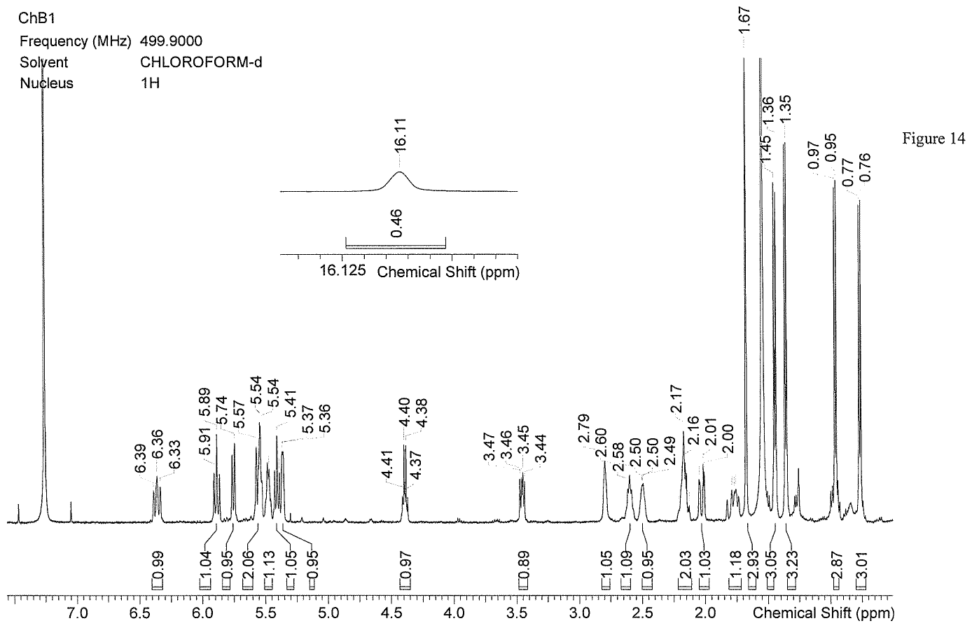

FIG. 14 shows the ¹H-NMR spectra of ChB1.

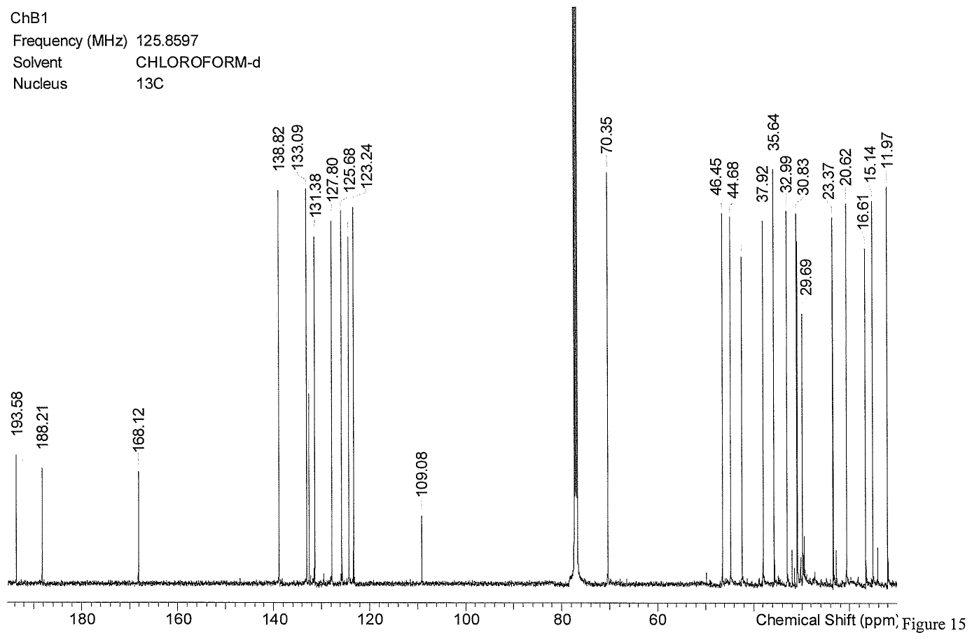

FIG. 15 shows the ¹³C-NMR spectra of ChB1.

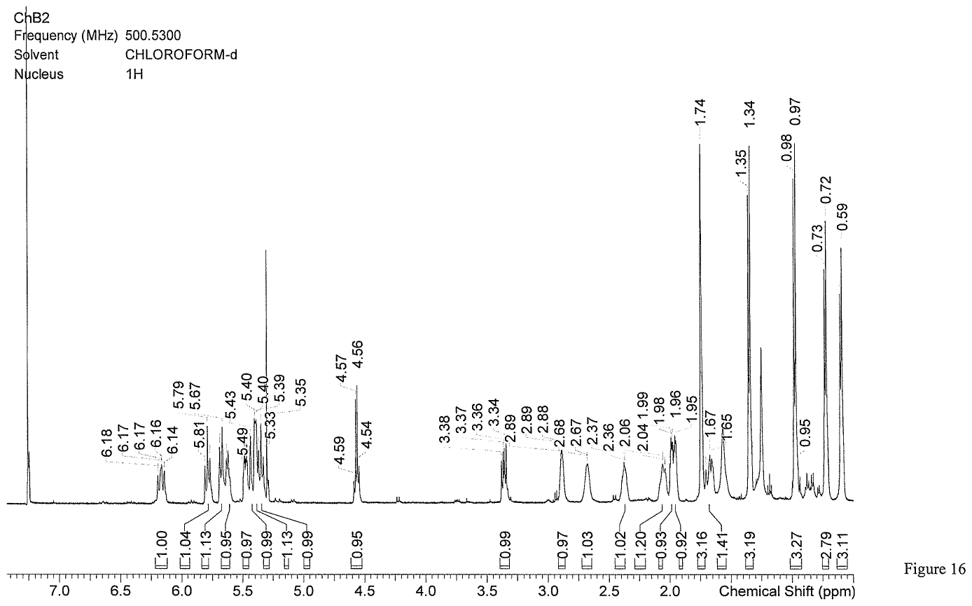

FIG. 16 shows the ¹H-NMR spectra of ChB2.

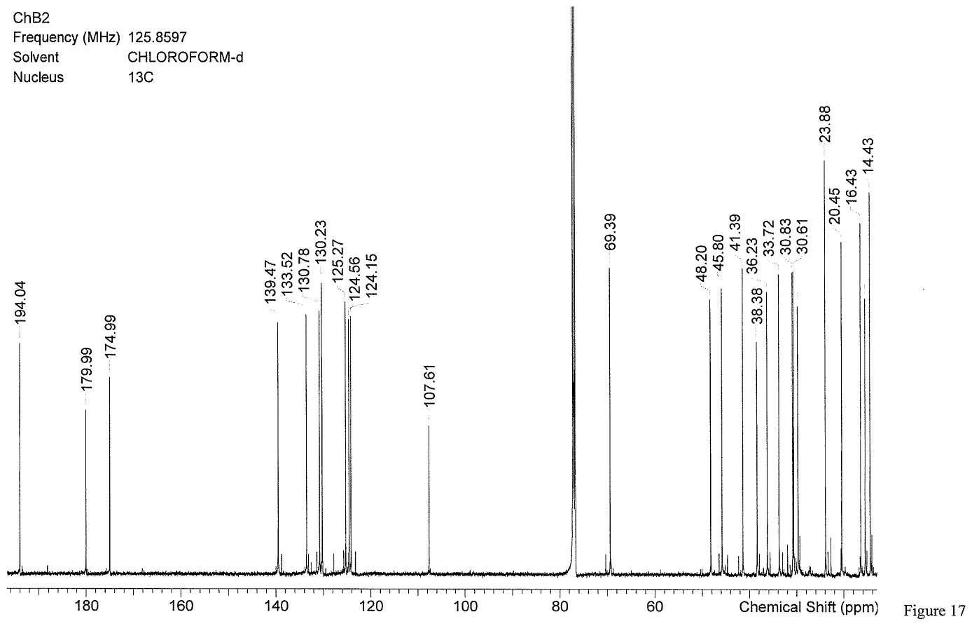

FIG. 17 shows the ¹³C-NMR spectra of ChB2.

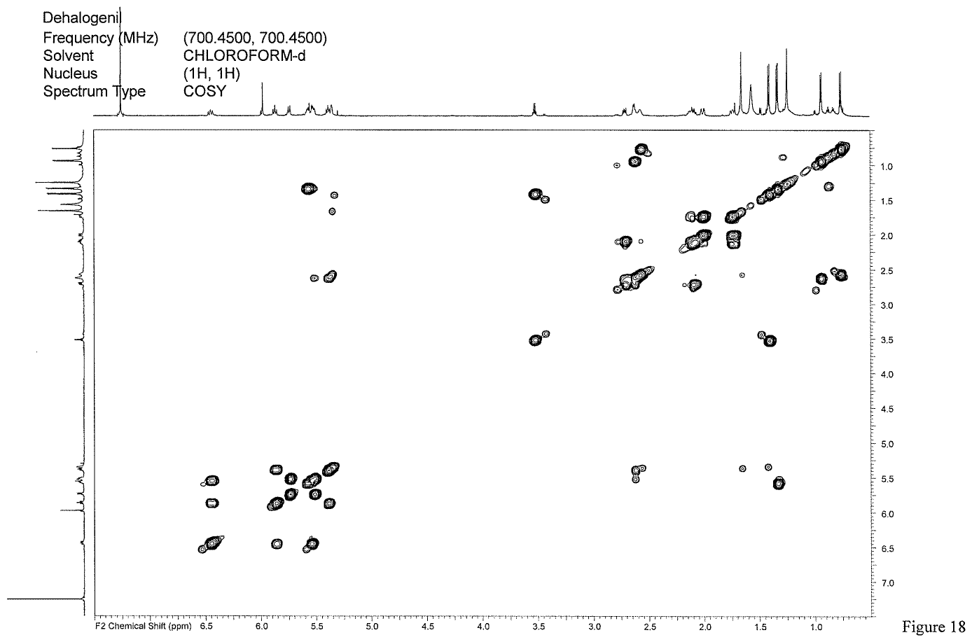

FIG. 18 shows the ¹H-¹H COSY spectrum of dehalogenil.

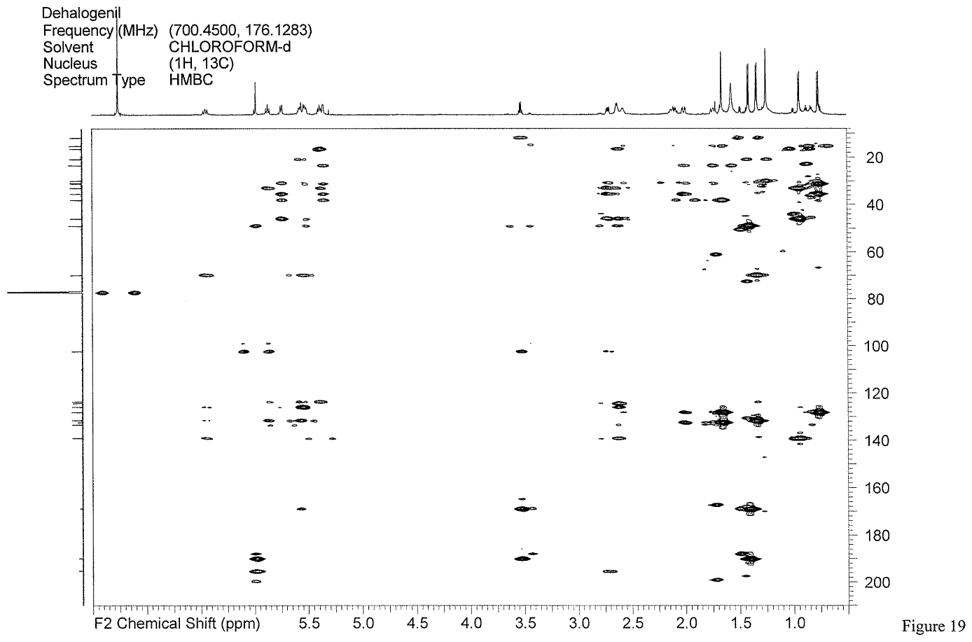

FIG. 19 shows the ¹H-¹³C HMBC spectrum of dehalogenil

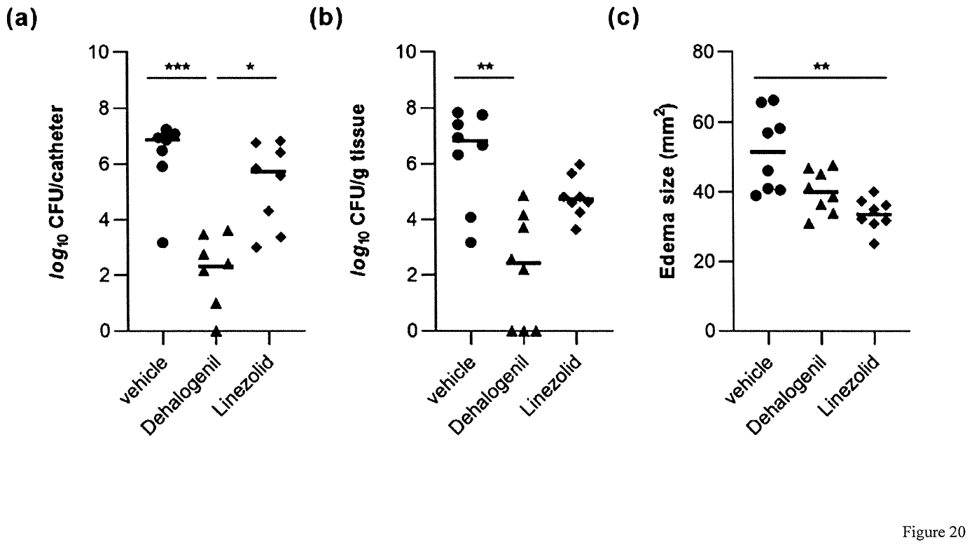

FIG. 20 shows the activity of dehalogenil in a S. aureus foreign-body infection model

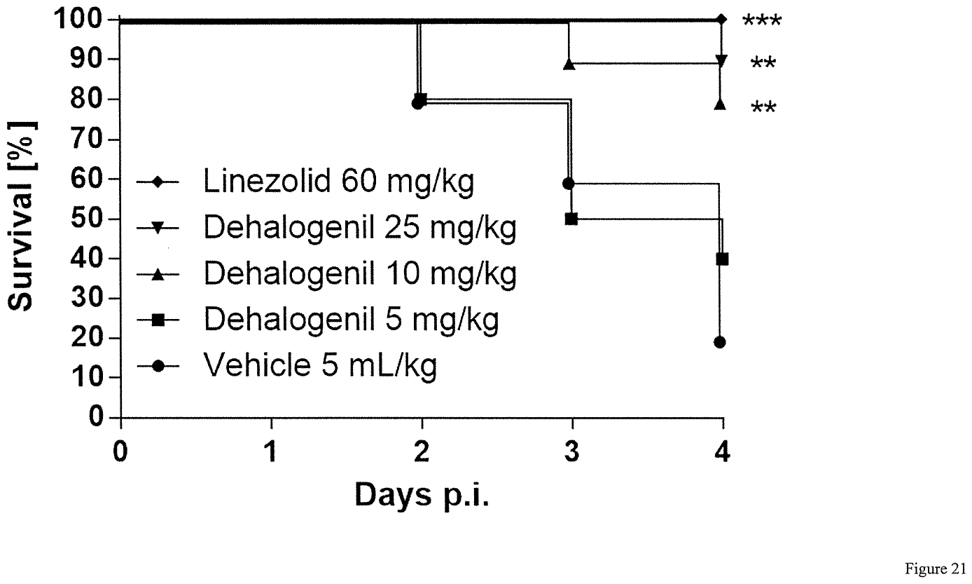

FIG. 21 shows the efficacy of dehalogenil in a S. aureus sepsis model

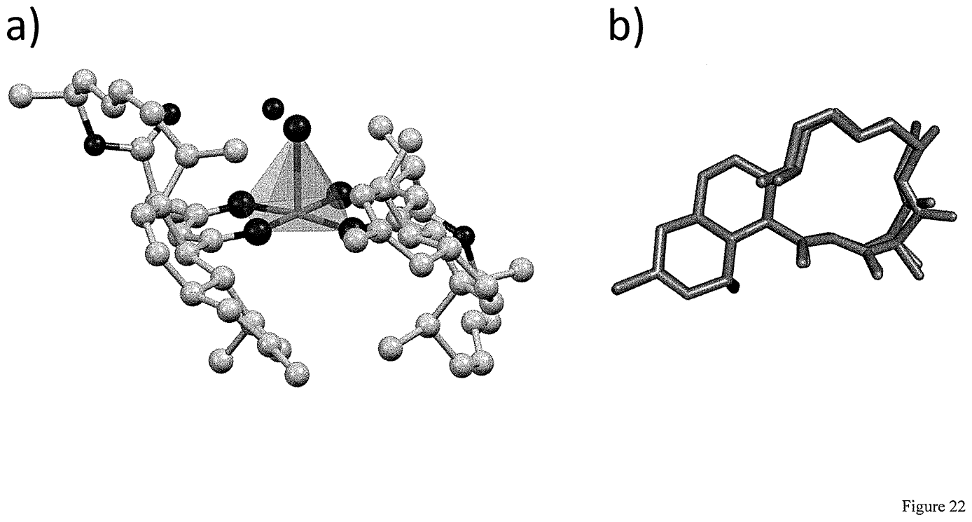

FIG. 22 shows the crystal structure of dehalogenil in coordination with copper (a) and the overlay of geometries of two independent molecules (b)

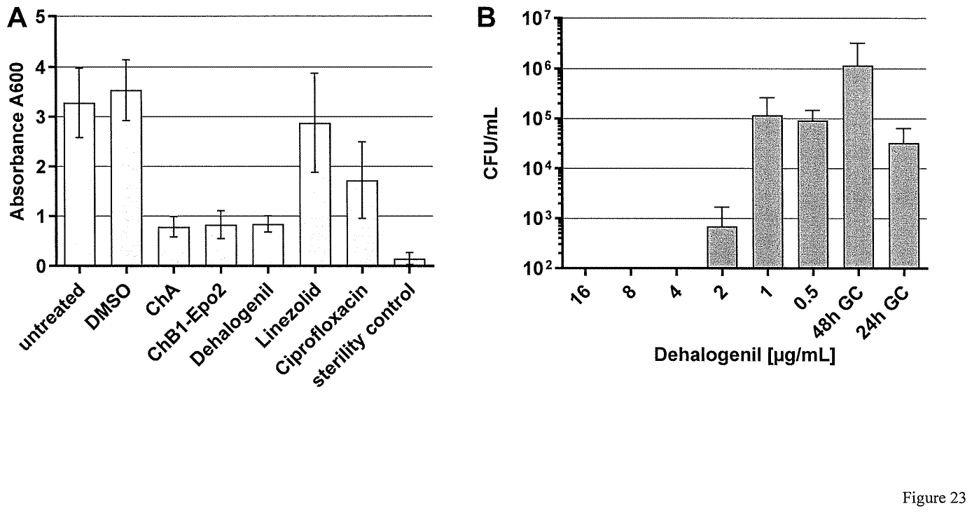

FIG. 23 shows the activity of chlorotonils against S. epidermidis biofilms

### DESCRIPTION OF DEHALOGENIL (WH0165_4) AND COMPARISON WITH ANTHRACIMYCIN

The high resolution mass (HRMS) of dehalogenil prepared by the method of the present invention is matching with the expected HRMS. Furthermore, no chlorine isotopic pattern is observed, indicating no isomer of chlorotonil. In the ¹H-NMR spectra, the most prominent peaks are a broad singlet at ca. 15.5 ppm and a singlet at ca. 6 ppm. These resemble the proton at the carbon between the 1,3-diketone and between the two oxygens of the 1,3-diketone. The shifts of most of the protons as well as carbons are closely matching with the corresponding positions of its isomer/epimer anthracimycin (Emma K. Davison et al. Org. Lett. 2020, 22, 14, 5550-5554).

Comparison of the Structure of Dehalogenil (WH0165_4) with the Published Product from the Total Synthesis^{1,2}

No sample was available for analysis of the published product obtained by total synthesis. For comparison, the data of the publication of the total synthesis¹ are used, which are in accordance to the PhD thesis². The HRMS in the publication of the total synthesis shows the correct value. Nevertheless, traces of the correct MS of dehalogenil can also be found as fragment in the mass spectrum of chlorotonil B1/B2. When the NMR spectra are compared, a singlet at ca. 16.1 ppm is found deviating strongly from WH0165_4. Furthermore, the remaining peaks have a strong shift of the signals from each other, especially if the position next to the carbonyl of the ester is compared (4.4 ppm vs 3.54 ppm). Also, no singlet for the proton at the carbon between the 1,3-diketone can be seen at 5.98 ppm although it is indicative for dehalogenil. When the proposed structure with two protons attached to the carbon between the diketone is used (see FIG. 1, dehalogenil described in the literature), a singlet or doublet should be seen which is not the case in the corresponding spectrum. Furthermore, no 2D-spectra and no assignment of the peaks were done in the literature, so the assignment especially of the protons in the aromatic range is difficult, as the two before mentioned protons will be in the aromatic range. Additionally, when the published product of the total synthesis is compared with ChB2, a strong match is observed suggesting that the published compound obtained from the total synthesis was ChB2, rather than the dehalogenated chlorotonil.

Comparison of the Structure of Dehalogenil (WH0165_4) with the Product after Dehalogenation with Ascorbic Acid/Triethylamine (WH0236A)³

No sample was available for analysis of the published product obtained after dehalogenation by ascorbic acid/triethylamine. Therefore, ChA was dehalogenated as described³ by the inventors to obtain the reference material. The mass in the thesis³ shows the correct, expected value. A peculiar observation is that the mass was described as sodium adduct in contrast to the proton adduct from the total synthesis^{1,2} showing already deviation of the products of the total synthesis and the dehalogenation of ChA. When the NMR spectra are compared, the spectrum of WH0165_4 does not match with the spectrum given in the PhD thesis³. Instead, the resynthesis by the inventors yielded ChB1. Additionally, the ¹H-NMR spectrum from the PhD thesis³ also deviates from the spectrum reported for the total synthesis^{1,2}. Interestingly, the ¹³C-NMR reported from the total synthesis^{1,2} is in accordance with the spectrum reported in the PHD thesis³. In the case of the resynthesis by the inventors (WH0236A), ChB1 was obtained rather than the dehalogenated compound. A comparison between the reported data of the PHD thesis³ and ChB2 shows that most likely ChB2 was obtained in the PHD thesis because all peaks of the ¹H- and ¹³C-NMR spectrum are matching in the NMR spectrum. The fact that ChB2 is the salt of ChB1 also underlines that the singlets at ca. 16.1 ppm and ca. 6.0 ppm are missing. Furthermore, no 2D-spectra and no assignment of the peaks were done in the PhD thesis³ so the assignment especially of the protons in the aromatic range is difficult.

Comparison of the Structure of Dehalogenil (WH0165_4) with the Product after Dehalogenation with SmI₂ (WH0236B)²

The NMR and mass data of this dehalogenation were reported identically in the PhD thesis² as for the total synthesis¹. Therefore, the analysis of the reported data is the same as described above for the total synthesis. No sample was available for analysis of the published product obtained after dehalogenation by SmI₂. Therefore, ChA was dehalogenated as described² by the inventors to obtain the reference material. As in case of dehalogenation under the conditions of WH0236A, the same compound was obtained by dehalogenation with SmI₂. ChB1 was therefore obtained in all cases of the semi synthetic dehalogenation reactions previously described. The small differences between the reported ¹H spectra could be due to different conditions in the NMR; nevertheless, the ¹³C-NMR reveals that the structure reported was ChB2. Since the synthesis of WH0236B led to ChB1, the published NMR and mass data are incorrect and no dehalogenil was obtained.

### Conclusion

All three known methods to obtain dehalogenated chlorotonil were investigated or discussed. The dehalogenation by ascorbic acid/triethylamine by the inventors led to ChB1 in contrast to the claimed dehalogenil but the reported spectra of the thesis resemble ChB2. The dehalogenation with SmI₂ by the inventors led to ChB1 and the spectra in the literature is similar to ChB2 and not the claimed dehalogenil. Therefore, the resynthesis with SmI₂ or ascorbic acid/triethylamine proved to be not reproducible and gave neither in the literature nor by resynthesis of the inventors dehalogenil. Furthermore, the ¹³C-NMR indicated that ChB2 could be formed in case of the formerly described synthesis.^{1,2} As these structures are the same as for the total synthesis, no dehalogenated chlorotonil has been described so far. For the biological evaluation, it can also be concluded that ChB2 was used rather than dehalogenil. In summary, the new dehalogenation route of the present invention yields dehalogenil for the first time.

### Experimental Procedures

### Dehalogenil (WH0165_4)

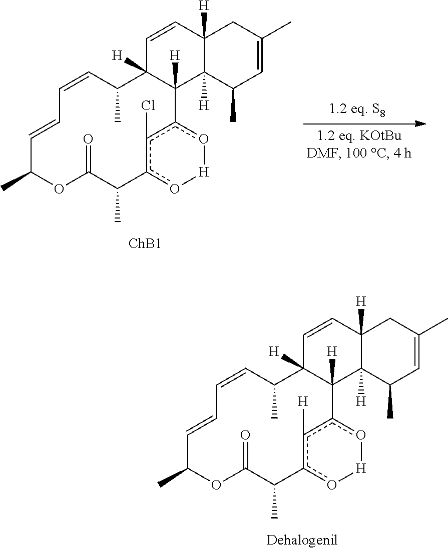

ChB1 (10.0 mg; 23 μmol), 1.2 eq. sulfur (0.9 mg; 27 μmol) and 1.2 eq. KOtBu (3.0 mg; 27 μmol) were dissolved in 1 ml dry DMF. The reaction was stirred 4 h at 100° C. The mixture was extracted three times with 10 ml DCM from 10 ml water. The combined organic phases were dried over MgSO₄ and the solvent was evaporated. The reaction was purified by flash chromatography over silica preequilibrated in hexane with an eluent gradient of DCM:Hex 1:5-2:5-3:2-DCM. Yield: 2.4 mg, 6 μmol, 26%, pale yellow powder. R_{f} (Silica, DCM:Hex 3:2): 0.15. HRMS: C₂₆H₃₅O₄ [M+H]⁺: calc.: 411.25299, found: 411.2536; Δ=1.48 ppm.

### Former Description of ChA Dehalogenation

### WH0236A

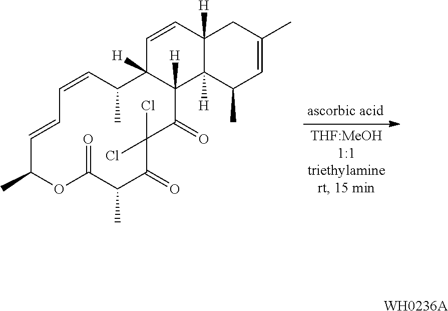

To Chlorotonil A (10.0 mg; 0.02 mmol) under nitrogen atmosphere in a 25 ml round bottom flask in 2 ml THF:MeOH 1:1, 100 mg ascorbic acid in 6 ml MeOH/triethylamine 1:1 was added. The reaction was stirred for 15 min at rt. The reaction was cooled to 0° C. and 3 ml acidic acid was added. The solution was warmed to rt and water was added. The solution was extracted with MTBE three times. The combined organic phases were washed with sat. bicarbonate solution and brine. The organic solvent was dried over MgSO₄ and the solvent was evaporated. The non-purified sample was evaluated by high resolution MS and ¹H-NMR.

### WH0236B

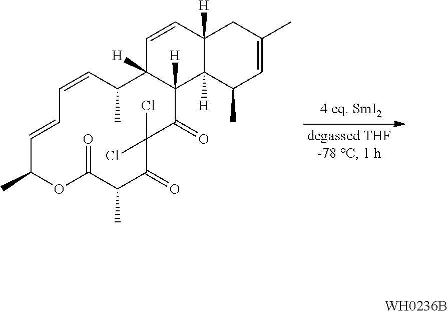

ChA (10.0 mg; 0.02 mmol) was dissolved in degassed 0.3 ml THF/MeOH. The solution was cooled to −78° C. and 4 eq. of SmI₂ (33.9 mg; 0.08 mmol; 405.73 g/mol; 840 μl) was added. The solution was stirred at −78° C. for one hour. The reaction was quenched with 1.5 ml EtOAc and 1 ml 0.1 M HCl. The solution was diluted with water and extracted three times with EtOAc. The combined organic phases were washed with sat. aq. NaHCO₃ solution and the organic layer was dried over MgSO₄. The solvent was evaporated. The non-purified sample was evaluated by high resolution MS and ¹H-NMR.

### Methods

### LCMS and LCMSMS Conditions

The measurements to detect all chlorotonil derivatives in high resolution were performed on a Dionex Ultimate 3000 RSLC system using a BEH C18, 50×2.1 mm, 1.7 μm dp column (Waters, Germany). Separation of 1 μl sample was achieved by a linear gradient from (A) H₂O+0.1% FA to (B) ACN+0.1% FA at a flow rate of 600 μL/min and 45° C. The gradient was initiated by a 0.5 min isocratic step at 5% B, followed by an increase to 95% B in 6 min to end up with a 2 min step at 95% B before re-equilibration under the initial conditions. UV spectra were recorded by a DAD in the range from 200 to 600 nm. The LC flow was split to 75 μL/min before entering the maXis 4G hr-TOF mass spectrometer (Bruker Daltonics, Germany) using the Apollo ESI source. Mass spectra were acquired in centroid mode ranging from 150-2500 m/z at a 2 Hz scan rate. Settings for MS/MS measurements were: minimum precursor intensity is set to 10000. Full scan spectra are acquired at 2 Hz followed by MS/MS spectra acquisition at variable scan speed ranging from 1 to 3 Hz, as a function of precursor intensity. CID energy varies linearly from 30, 35, 45, to 55 eV with respect to the precursor m/z from 300, 600, 1000, to 2000 m/z. The collision cell is set to ramp collision energy (80-120% of the set value with equal weights of both values), collision RF (700 to 1000 Vpp with equal weights of both values) and transfer time (90-110 μs) for every MS/MS scan. The number of precursor was set to 2 and precursors were moved to an exclusion list for 0.2 min after two spectra were measured (typical chromatographic peak width was 0.10-0.15 min). Precursors were reconsidered if their intensity changed fivefold.

### NMR Measurements

All 1D (¹H and ¹³C) NMR spectra were recorded on a Bruker Ascend 700 spectrometer with a 5 mm TXI cryoprobe (¹H at 700 MHz, ¹³C at 175 MHz) or Bruker Avance Neo 500 MHz, equipped with a Prodigy Cryo-probe. The samples were dissolved in CDCl₃ and the chemical shifts of the solvent signals at 7.26 ppm (8H) and 77.16 ppm (8° C.) were considered as internal standard (reference signal). The observed chemical shift (8) values are given in ppm and the coupling constants (J) in Hz.

### Crystallography

In an attempt to grow dehalogenil (DH) nanocrystals suitable for electron diffraction analysis, a carbon-coated copper TEM grid was dipped directly into 2 ml of ethanol suspension of dehalogenil. A little hole was punched in the plastic lid of the vial. The solvent would then slowly evaporate from the vial through the hole, and eventually crystals of DH would grow on the bottom and on the TEM grid. When the solvent was evaporated, it turned out that dehalogenil incorporated Cu²⁺ ions from the grid. The crystal was mounted at room temperature with perfluoroether oil on top of a CryoLoop (Hampton Research) and placed in a cold nitrogen gas stream on an XtaLAB Synergy (Rigaku) diffractometer equipped with micro-focus PhotonJet (Cu) X-ray Source. Data collection and structure relevant data are presented below.

Crystal data

| Chemical formula | C₅₂H₆₆CuO₈•0.40(H₂O)•0.42(H₂O) |
|---|---|
| Mᵣ | 897.35 |
| Crystal system, space group | Orthorhombic, P212121 |
| Temperature (K) | 103 |
| a, b, c (Å) | 11.0991 (1), 16.4287 (2), 26.8500 (3) |
| V (Å³) | 4895.93 (9) |
| Z | 4 |
| Radiation type | Cu K_{α} |
| μ (mm⁻¹) | 1.05 |
| Crystal size (mm) | 0.18 × 0.17 × 0.12 |

Data collection

| | |
|---|---|
| Diffractometer | XtaLAB Synergy, Single source at |
| | home/near, HyPix |
| Absorption correction | Multi-scan |
| | CrysAlis PRO 1.171.42.69a (Rigaku |
| | Oxford Diffraction, 2022) |
| | Empirical absorption correction using |
| | spherical harmonics, implemented in |
| | SCALE3 ABSPACK scaling algorithm. |

### 2′Demethyldehalogenil (Dehalogenil-2′DM)

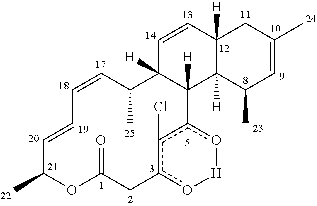

Isolation from a large-scale fermentation, white amorphous solid.

HRMS: C₂₅H₃₃O₄ [M+H]⁺: calc.: 397.23734, found: 397.23795; Δ=1.55 ppm.

### NMR Table of 2′-Demethyldechlorotonil

| H | δ_{H} | m | J(Hz) | COSY | C | δ_{C} | HMBC |
|---|---|---|---|---|---|---|---|
| — | | | | | 1 | 165.7 | 2, 21, 4 |
| 2α | 3.23 | d | 11.5 | 2β | 2 | 46.4 | 4 |
| 2β | 3.49 | d | 11.5 | 2α | | | |
| — | | | | | 3 | 184.3 | 2, 4 |
| 4 | 5.98 | s | | 2β, 2α | 4 | 102.5 | 6, 2 |
| — | | | | | 5 | 197.8 | 6, 4, 2β |
| 6 | 2.74 | dd | 11.9, 7.0 | 12, 7, 15, 11, 14 | 6 | 49.6 | 7, 8, 6, 14, 4, 23 |
| 7 | 2.10 | m | | 12, 11, 8, 6, 13 | 7 | 35.4 | 23, 8, 11, 6, 9, 13 |
| 8 | 2.60 | m | | 23, 24, 12, 7, 9 | 8 | 31.0 | 23, 9, 6, 7, 11 |
| 9 | 5.35 | dt | 2.7, 1.2 | 24, 11, 8, 23 | 9 | 128.2 | 23, 8, 11, 7 |
| — | | | | | 10 | 132.5 | 11 |
| 11α | 2.00 | m | | 24, 11β, 12, 7, 9 | 11 | 38.2 | 24, 11, 9, 13 |
| 11β | 1.70 | m | | 24, 11α, 12, 7, 9 | | | |
| 12 | 2.10 | m | | 7, 11, 8, 6, 13 | 12 | 44.1 | 11, 7, 13, 14, 9 |
| 13 | 5.74 | d | 10.0 | 12, 15, 21, 14 | 13 | 133.6 | 18, 14, 15 |
| 14 | 5.52 | m | | 13, 15, 6 | 14 | 124.3 | 16, 18, 13, 17 |
| 15 | 2.65 | ddq | 6.8, 4.5, 2.2 | 6, 16, 14, 17, 7 | 15 | 45.7 | 25, 18, 14, 13 |
| 16 | 2.61 | m | | 17, 15, 18, 25 | 16 | 33.0 | 25, 15, 6, 17, 18 |
| 17 | 5.39 | dd | 10.3, 8.8 | 16, 18, 19 | 17 | 139.2 | 25, 16, 19 |
| 18 | 5.88 | m | | 17, 19, 20, 16, 25 | 18 | 126.0 | 25, 19, 20, 16 |
| 19 | 6.47 | m | | 22, 20, 18, 17 | 19 | 123.7 | 22, 20, 18 |
| 20 | 5.56 | m | | 22, 21, 19 | 20 | 131.5 | 22, 17, 18, 19 |
| 21 | 5.55 | m | | 22, 20, 19 | 21 | 70.1 | 22, 2, 17, 19 |
| 22 | 1.35 | d | 6.6 | 21, 20, 19 | 22 | 44.1 | 20, 21 |
| 23 | 0.74 | d | 7.1 | 8, 23 | 23 | 43.9 | 24, 11, 7 |
| 24 | 1.66 | d | 0.6 | 9, 11, 8 | 24 | 44.0 | 11, 9 |
| 25 | 0.94 | d | 6.6 | 16, 18 | 25 | 43.9 | 16, 17 |

### 2′Demethylchlorotonil (ChB1-2′DM)

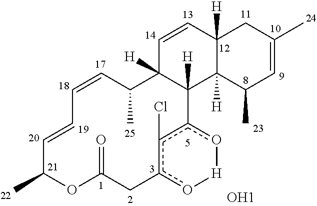

Isolation from a large-scale fermentation, white amorphous solid.

HRMS: C₂₅H₃₂ClO₄ [M+H]⁺: calc.: 431.1984, found: 431.1986; Δ=−0.5 ppm.

### NMR Table of 2′-Demethylchlorotonil

| H | δ_{H} | m | J(Hz) | COSY | C | δ_{C} | HMBC |
|---|---|---|---|---|---|---|---|
| — | | | | | 1 | 164.8 | 2, 2 |
| 2β | 4.24 | d | 13.1 | 2β, OH1 | 2 | 43.6 | OH1 |
| 2α | 3.27 | d | 13.1 | 2α | | | |
| — | | | | | 3 | 182.4 | 2, 2, OH1 |
| — | | | | | 4 | 109.3 | 2, 2, OH1 |
| — | | | | | 5 | 195.5 | 6, OH1 |
| 6 | 3.47 | br dd | 11.7, 6.7 | 7, 15 | 6 | 46.7 | 7, 14, OH1 |
| 7 | 2.15 | m | | 8, 15, 6 | 7 | 35.6 | 23, 11, 8, 6, 9, 13 |
| 8 | 2.50 | m | | 23, 24, 7, 9 | 8 | 30.7 | |
| 9 | 5.36 | br s | | 24, 8 | 9 | 127.7 | 23, 24, 11 |
| — | | | | | 10 | 132.5 | 24, 11 |
| 11α | 2.03 | m | | 11β, 12 | 11 | 37.9 | 24, 7, 9, 13 |
| 11β | 1.73 | m | | 11α, 12 | | | |
| 12 | 2.18 | m | | 11 | 12 | 30.7 | 11, 7, 8, 6, 9, 13 |
| 13 | 5.76 | br d | 10.4 | 15, 14 | 13 | 133.2 | 15 |
| 14 | 5.55 | m | | 15, 13 | 14 | 124.1 | 16, 15, 13 |
| 15 | 2.81 | m | | 7, 6, 14, 13 | 15 | 42.3 | 25, 16, 6, 14, 13 |
| 16 | 2.59 | m | | 25, 17 | 16 | 33.1 | 25, 6 |
| 17 | 5.42 | m | | 16, 20 | 17 | 138.9 | 25, 16, 19 |
| 18 | 5.60 | br d | 2.3 | 19 | 18 | 131.1 | 22, 17, 21, 20, 19 |
| 19 | 6.42 | m | | 18, 20 | 19 | 123.2 | 22, 17, 20 |
| 20 | 5.91 | m | | 17, 19 | 20 | 125.7 | 18, 19 |
| 21 | 5.47 | m | | 22 | 21 | 70.7 | 22, 19 |
| 22 | 1.39 | d | 6.6 | 21 | 22 | 20.5 | 21 |
| 23 | 0.75 | d | 7.0 | 8 | 23 | 15.1 | 7 |
| 24 | 1.68 | s | | 8, 9 | 24 | 23.4 | 11, 9 |
| 25 | 0.97 | d | 6.6 | 16 | 25 | 16.5 | 16, 15, 17 |
| OH1 | 15.85 | s | | 2 | — | | |

### ChA-4′F

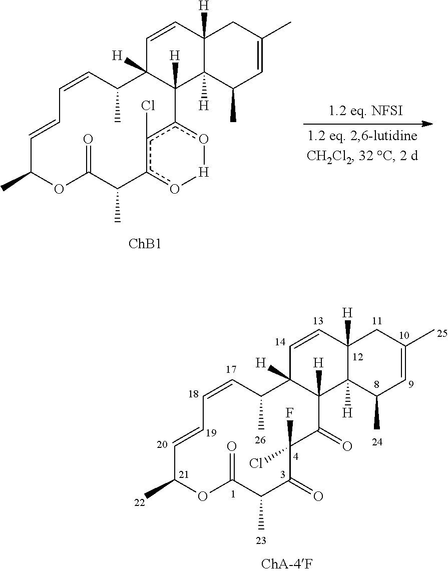

ChB1 (10.0 mg; 23 μmol) and 1.2 eq. NFSI (8.5 mg; 27 μmol) were dissolved in 1 mL dry CH₂Cl₂ followed by addition of 1.2 eq. 2,4-lutidine (2.9 mg; 27 μmol; 3.1 μl; one drop). The reaction was stirred for three days at 32° C. The mixture was extracted three times with 10 mL CH₂Cl₂ from 10 mL water. The combined organic phases were dried over MgSO₄ and the solvent was evaporated. The reaction was purified by flash chromatography over silica preequilibrated in hexane with an eluent gradient of CH₂Cl₂:Hex 0.5:5-1:5-1.5:5-2:5-3:2-CH₂Cl₂. Yield: 2.3 mg; 5 μmol; 22%. R_{f} (Silica, CH₂Cl₂:Hex 3:2): 0.5. HRMS: C₂₆H₃₃ClFO₄ [M+H]⁺: calc.: 463.20459, found: 463.20439; Δ=0.43 ppm.

### NMR Table of ChA-4′F

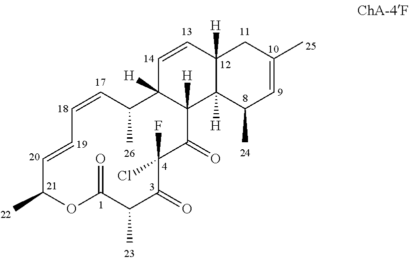

ChA-4′F

| H | δ_{H} | m | J(Hz) | COSY | C | δ_{C} | HMBC |
|---|---|---|---|---|---|---|---|
| — | | | | | 1 | 168.0 | 23, 2, 21 |
| 2 | 4.46 | q | 7.2 | 23 | 2 | 47.3 | 23 |
| — | | | | | 3 | 193.9 | 23, 2 |
| 4 | | | | | 4 | — | — |
| — | | | | | 5 | 194.1 | — |
| 6 | 3.71 | dd | 11.5, 6.7 | 7, 15 | 6 | 49.7 | 12, 14 |
| 7 | 2.16 | m | | 6 | 7 | 36.5 | 11, 8, 6, 9, 13 |
| 8 | 2.41 | m | | 24 | 8 | 30.1 | 24, 11, 7, 9, 12 |
| 9 | 5.35 | m | | 25, 8 | 9 | 127.9 | 25, 11 |
| — | | | | | 10 | 132.5 | 11 |
| 11α | 2.03 | br dd | 16.8, 4.0 | 11β, 12 | 11 | 38.2 | 12, 9, 13 |
| 11β | 1.75 | m | | 11α, 12 | | | |
| 12 | 2.15 | m | | 11α, 11β | 12 | 30.8 | 8, 6, 14, 13 |
| 13 | 5.74 | br d | 9.9 | 14 | 13 | 133.3 | 6, 14 |
| 14 | 5.53 | m | | 15, 13 | 14 | 123.8 | 16, 17, 13 |
| 15 | 2.99 | m | | 6, 14 | 15 | 43.2 | 26, 16, 6, 14, 13, 18 |
| 16 | 2.85 | m | | 26, 17 | 16 | 33.1 | 26, 15, 6, 17, 18 |
| 17 | 5.34 | m | | 16, 18 | 17 | 139.4 | 16, 20, 19 |
| 18 | 5.88 | m | | 17, 19 | 18 | 125.2 | 26, 20, 21, 19, 19 |
| 19 | 6.06 | m | | 20, 18 | 19 | 125.4 | 18, 18 |
| 20 | 5.52 | m | | 22, 19 | 20 | 130.5 | 21, 18, 19 |
| 21 | 5.61 | m | | 22 | 21 | 70.6 | 11, 20, 19 |
| 22 | 1.34 | s | | 20, 21 | 22 | 21.0 | 23, 20, 21 |
| 23 | 1.49 | d | 7.1 | 2 | 23 | 14.5 | 7, 2 |
| 24 | 0.72 | d | 7.1 | | 24 | 14.7 | — |
| 25 | 1.67 | s | | 9 | 25 | 23.4 | 11, 9 |
| 26 | 0.95 | d | 6.6 | 16 | 26 | 16.2 | 17 |

### ChA-4′Me

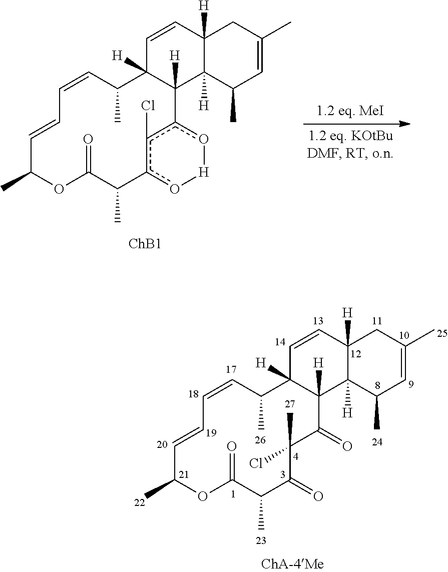

ChB1 (10.0 mg; 23 μmol) and 1.2 eq. KOtBu (3.0 mg; 27 μmol) was dissolved in 1 mL dry DMF. After 5 min, 1.2 eq. Mel (3.8 mg; 27 μmol; 1.9 μl (one drop)) was added. The reaction was stirred over night at room temperature. The mixture was extracted three times with 10 mL CH₂Cl₂ from 10 mL water. The combined organic phases were dried over MgSO₄ and the solvent was evaporated. The reaction was purified by flash chromatography over silica preequilibrated in hexane with an eluent gradient of CH₂Cl₂:Hex 1:5-2:5-3:2-CH₂Cl₂. Yield: 4.9 mg; 11.1 μmol; 49%. R_{f} (Silica, CH₂Cl₂:Hex 3:2): 0.19. HRMS: C₂₇H₃₆ClO₄ [M+H]⁺: calc.: 459.22966, found: 459.22925; Δ=0.89 ppm.

### NMR Table of ChA-4′Me

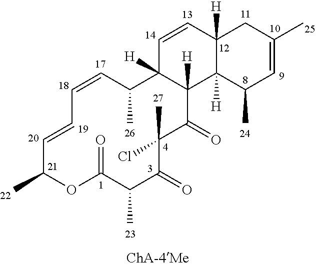

ChA-4′Me

| H | δ_{H} | m | J(Hz) | COSY | C | δ_{C} | HMBC |
|---|---|---|---|---|---|---|---|
| — | | | | | 1 | 168.4 | 23, 2, 21 |
| 2 | 4.46 | q | 7.0 | 23, 27 | 2 | 46.8 | 23 |
| — | | | | | 3 | 198.3 | 23, 27, 2 |
| 4 | | | | | 4 | 77.7 | 27 |
| — | | | | | 5 | 203.8 | 27, 6 |
| 6 | 3.72 | m | | 11α, 11β, 7, 15 | 6 | 49.5 | 7, 15, 14 |
| 7 | 2.16 | m | | 11, 12, 8, 6, 13 | 7 | 36.8 | 24, 11, 12, 8, 6, 9, 13 |
| 8 | 2.30 | br d | 4.3 | 24, 25, 7, 9 | 8 | 30.3 | 24, 22, 11, 9, 13, 12 |
| 9 | 5.37 | dt | 2.8, 1.3 | 25, 11, 8 | 9 | 128.1 | 24, 11 |
| — | | | | | 10 | 132.6 | 11 |
| 11α | 2.03 | dd | 17.0, 4.0 | 25, 11β, 12, 6 | 11 | 38.6 | 25, 9, 13 |
| 11β | 1.75 | m | | 25, 11α, 12, 7, 6, 9 | | | |
| 12 | 2.16 | m | | 11α, 11β, 7, 13 | 12 | 30.5 | 7, 8, 6 |
| 13 | 5.71 | d | 10.1 | 7, 15, 14, 12 | 13 | 132.9 | 15, 14 |
| 14 | 5.51 | m | | 15, 13 | 14 | 130.3 | 11, 9 |
| 15 | 3.07 | dtd | 6.3, 4.2, 1.9 | 16, 6, 14, 13 | 15 | 42.1 | 26, 6, 14, 13 |
| 16 | 2.85 | m | | 26, 15, 17, 18 | 16 | 33.3 | 15, 6, 17 |
| 17 | 5.28 | td | 9.2, 0.6 | 16, 20, 18, 19 | 17 | 139.7 | 26, 16, 19 |
| 18 | 5.88 | m | | 16, 17, 20, 19 | 18 | 125.5 | 20, 19 |
| 19 | 6.07 | m | | 17, 20, 21, 18 | 19 | 124.0 | 17, 20, 21, 18 |
| 20 | 5.51 | s | | 17, 21, 18, 19 | 20 | 124.2 | 22, 19 |
| 21 | 5.65 | q | 6.7 | 22, 20, 19 | 21 | 69.9 | 22, 14, 19 |
| 22 | 1.32 | d | 6.9 | 21 | 22 | 21.0 | 21 |
| 23 | 1.48 | d | 7.2 | 2 | 23 | 16.5 | 22, 2 |
| 24 | 0.76 | d | 7.2 | 8 | 24 | 15.1 | 25, 7 |
| 25 | 1.66 | s | | 11α, 11β, 8, 9 | 25 | 23.4 | 9 |
| 26 | 0.96 | d | 6.4 | 16 | 26 | 15.9 | 15, 17 |
| 27 | 1.95 | s | | 2 | 26 | 28.2 | |

### ChB1-2′Me

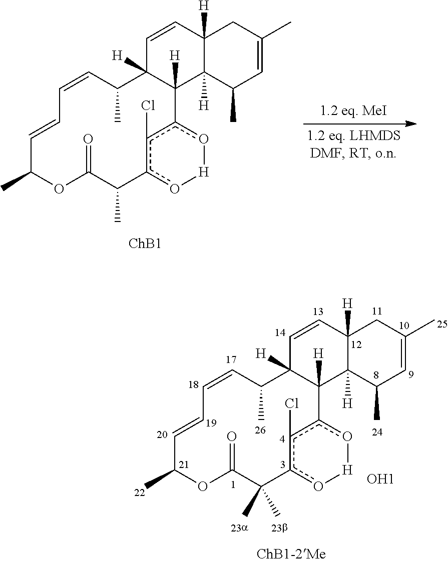

ChB1 (10.0 mg; 23 μmol) was dissolved in 1 mL dry DMF and 1.2 eq. LHMDS (1 M in THF; 27 μl) was added. After 5 min, 1.2 eq. Mel (3.8 mg; 27 μmol; 1.9 μl (one drop)) was added. The reaction was stirred over night at room temperature. The mixture extracted three times with 10 mL CH₂Cl₂ from 10 mL water. The combined organic phases were dried over MgSO₄ and the solvent was evaporated. The reaction was purified by flash chromatography over silica preequilibrated in hexane with an eluent gradient of CH₂Cl₂:Hex 0.5:5-1:5-1.5:5-2:5-3:2. Yield: 3.0 mg; 7 μmol; 29%. R_{f} (Silica, CH₂Cl₂:Hex 3:2): 0.45. HRMS: C₂₇H₃₆ClO₄ [M+H]⁺: calc.: 459.22966, found: 459.22956; Δ=0.23 ppm.

### NMR Table of ChB1-2′Me

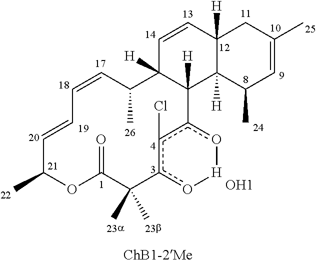

ChB1-2′Me

| H | δ_{H} | m | J(Hz) | COSY | C | δ_{C} | HMBC |
|---|---|---|---|---|---|---|---|
| — | | | | | 1 | 172.4 | 23β, 23α |
| 2 | | | | | 2 | 53.0 | 23β, 23α |
| — | | | | | 3 | 196.3 | 23β, 23α |
| 4 | | | | | 4 | 108.1 | 6 |
| — | | | | | 5 | 189.4 | 6 |
| 6 | 3.36 | dd | 11.5, 6.6 | 12, 7, 15 | 6 | 45.2 | 12, 7, 16, 14 |
| 7 | 2.17 | m | | 11, 12, 6 | 7 | 31.0 | 11α, 11β, 12, 8, 6, 13 |
| 8 | 2.47 | m | | 25, 24, 12, 9 | 8 | 31.2 | 25, 9 |
| 9 | 5.35 | m | | 24, 11, 8 | 9 | 128.0 | 25, 24, 11α, 11β, 8 |
| — | | | | | 10 | 132.7 | 24, 11α, 11β |
| 11α | 2.03 | br dd | 16.9, 4.2 | 24, 11, 7 | 11 | 38.2 | 25, 24, 12, 7, 9, 13 |
| 11β | 1.75 | m | | 24, 11, 12, 7, 9 | | | |
| 12 | 2.16 | m | | 11, 7, 8, 6 | 12 | 36.0 | 25, 11α, 11β 7, 8, 15, 6, 9, 13 |
| 13 | 5.74 | br d | 9.9 | 15, 14 | 13 | 133.1 | 15, 14, 20 |
| 14 | 5.50 | ddd | 10.0, 4.4, 1.9 | 15, 13 | 14 | 124.7 | 16, 13 |
| 15 | 2.69 | m | | 6, 14, 13 | 15 | 43.3 | 26, 16, 6, 14, 13 |
| 16 | 2.73 | m | | 26, 17 | 16 | 33.1 | 26, 15, 6, 17, 18 |
| 17 | 5.42 | t | 8.7 | 16, 18 | 17 | 139.6 | 26, 16, 20, 19 |
| 18 | 5.92 | t | 11.0 | 17, 19 | 18 | 126.2 | 16, 20, 19 |
| 19 | 6.54 | m | | 20, 18 | 19 | 123.5 | 21, 17, 18 |
| 20 | 5.61 | dd | 15.3, 3.0 | 21, 19 | 20 | 131.3 | 22, 21, 17, 18, 19 |
| 21 | 5.05 | m | | 22, 20 | 21 | 74.1 | 22, 20, 19 |
| 22 | 3.36 | dd | 11.5, 6.6 | 21 | 22 | 20.9 | 21, 19 |
| 23α | 2.17 | m | | 23β | 23α | 22.6 | 23β |
| 23β | 2.47 | m | | 23α | 23β | 24.9 | 23α |
| 24 | 5.35 | m | | 11, 8, 9 | 24 | 23.5 | 11α, 11β, 9 |
| 25 | | | | 8 | 25 | 15.4 | 24, 12, 8 |
| 26 | 2.03 | br dd | 16.9 | 16 | 26 | 17.1 | 16, 17 |
| OH1 | 16.66 | s | | | — | | |

### Dehalogenil

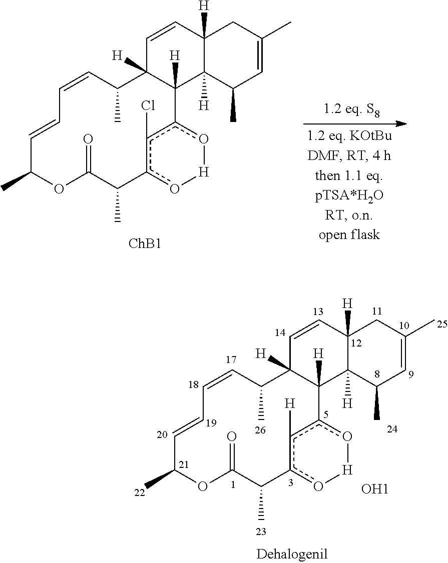

### Optimized Condition

ChB1 (68.9 mg; 155 μmol), 1.4 eq. sulfur (6.9 mg; 217 μmol) and 1.2 eq. KOtBu (21.0 mg; 186 μmol) were dissolved in 5 mL DMF. The reaction was stirred 5 h at RT in an open flask. Afterwards, 1.1 eq. TosOH*H₂O (32.4 mg; 171 μmol) was added and continued stirring for 1 d under the same conditions. The mixture was extracted three times with 50 mL CH₂Cl₂ from 50 mL water. The combined organic phases were dried over MgSO₄ and the solvent was evaporated. The reaction was purified by flash chromatography over silica preequilibrated in hexane with an eluent gradient of CH₂Cl₂:Hex 1:5-2:5-3:2-CH₂Cl₂. Yield: 45 mg, 110 μmol, 71%, pale yellow powder. Rr (Silica, CH₂Cl₂:Hex 3:2): 0.15. HRMS: C₂₆H₃₅O₄ [M+H]⁺: calc.: 411.25299, found: 411.2536; Δ=1.48 ppm.

### Dehalogenation by Photochemistry

A mixture of ChA (100 mg, 209 μmol), 0.1 eq. (Ir[dF(CF₃)ppy]₂(dtbpy))PF₆ (23.4 mg, 21 μmol), 5 eq. formic acid (48 mg, 39 μL, 1.00 mmol) and 5 eq. DIPEA (135 mg, 182 μL, 1.00 mmol) in 6 mL anhydrous DMF was purged with a balloon of nitrogen before being sealed in a vial. The reaction mixture was then stirred, under irradiation by blue LEDs without fan cooling (temperature reached ca. 35° C.), for 2 h. The crude reaction mixture was the submitted for purification via reverse phase prep-HPLC (acidic). Yield: 23 mg, 56 μmol, 27%, yellow/orange solid.

### NMR Table of Dehalogenil

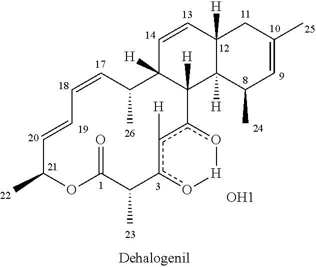

Dehalogenil

| H | δ_{H} | m | J(Hz) | COSY | C | δ_{C} | HMBC |
|---|---|---|---|---|---|---|---|
| — | | | | | 1 | 169.0 | 23, 2, 21 |
| 2 | 3.52 | q | 6.8 | | 2 | 49.0 | 23, 4 |
| — | | | | | 3 | 190.1 | 23, 2 |
| 4 | 5.98 | s | | | 4 | 102.4 | 6, 2 |
| — | | | | | 5 | 195.5 | 6 |
| 6 | 2.72 | dd | 12.0, 6.6 | 7, 12 | 6 | 49.0 | 8 |
| 7 | 2.09 | m | | 12, 6, 8 | 7 | 35.4 | 24, 11, 15, 6, 9, 13 |
| 8 | 2.58 | br d | 3.4 | 24, 9 | 8 | 31.1 | 24, 9, 12, 7 |
| 9 | 5.35 | br dd | 3.1, 1.4 | 25, 8 | 9 | 128.2 | 24, 25, 11, 8 |
| — | | | | | 10 | 132.5 | 25, 11 |
| 11α | 1.74 | m | | 11β, 12 | 11 | 38.2 | 25, 9, 12, 13 |
| 11β | 2.01 | br dd | 16.9, 4.5 | 11α | | | |
| 12 | 2.13 | m | | 11 | 12 | 30.8 | 11, 8, 6, 13 |
| 13 | 5.74 | br d | 10.0 | 14 | 13 | 133.5 | 15 |
| 14 | 5.51 | br dd | 6.1, 4.0 | 16, 15, 13 | 14 | 124.4 | 15, 16 |
| 15 | 2.63 | m | | 14 | 15 | 46.0 | 26, 16, 6, 14, 13 |
| 16 | 2.62 | m | | 26, 17, 14 | 16 | 32.9 | 26, 15, 17, 18 |
| 17 | 5.39 | br t | 9.4 | 16, 18 | 17 | 139.1 | 26, 16, 15, 19 |
| 18 | 5.86 | brt | 10.8 | 17, 19 | 18 | 126.0 | 16, 20, 19 |
| 19 | 6.44 | m | | 20, 18 | 19 | 123.7 | 17, 20, 21, 18 |
| 20 | 5.55 | m | | 19 | 20 | 131.7 | 22, 21, 18, 19 |
| 21 | 5.57 | m | | 22 | 21 | 69.9 | 22, 20, 19 |
| 22 | 1.34 | d | 6.8 | 21 | 22 | 21.0 | 21 |
| 23 | 1.41 | m | | 2 | 23 | 11.9 | 2 |
| 24 | 0.77 | m | | 8 | 24 | 15.4 | 8, 7 |
| 25 | 1.67 | s | | 9 | 25 | 23.5 | 11, 9 |
| 26 | 0.95 | d | 6.6 | 16 | 26 | 16.5 | 16, 17 |
| OH1 | 15.56 | br s | | | — | | |

### Dehalogenil-2′Epi

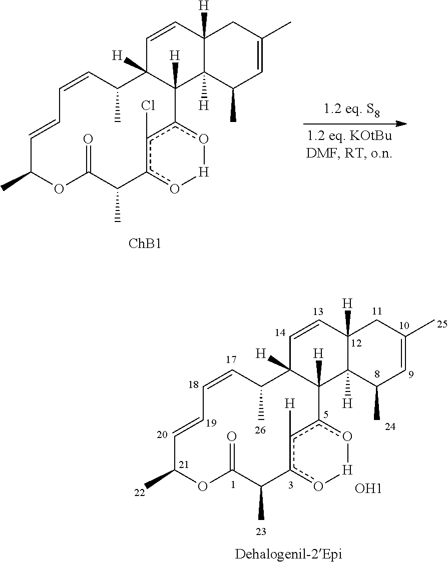

Dehalogenil-2′Epi was obtained from a dehalogenation reaction (see above; 70 mg of a dehalogenation reaction optimization mixture) by semi preparative HPLC. The gradient used was 30% ACN-95% ACN in 30 min. Yield: 2.4 mg; 6 μmol, white solid. R_{f} (Silica, CH₂Cl₂:Hex 3:2): 0.15. HRMS: C₂₆H₃₅O₄ [M+H]⁺: calc.: 411.25299, found: 411.25334; Δ=0.86 ppm.

### NMR Table of Dehalogenil-2′Epi

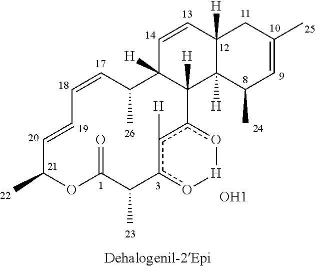

Dehalogenil-2′Epi

| H | δ_{H} | m | J(Hz) | COSY | NOESY | C | δ_{C} | HMBC |
|---|---|---|---|---|---|---|---|---|
| — | | | | | | 1 | 168.6 | 23, 2 |
| 2 | 3.45 | m | | 23 | 23 | 2 | 50.4 | 4 |
| — | | | | | | 3 | 187.8 | 23, 2, 4 |
| 4 | 6.00 | m | | | 23, 15, 6, 19 | 4 | 98.9 | 6, 2 |
| — | | | | | | 5 | 199.5 | 6, 4 |
| 6 | 2.79 | dd | 11.9, | 7, 12, 15 | 24, 12, 4 | 6 | 50.1 | 7 |
| | | | 7.0 | | | | | |
| 7 | 2.09 | m | | 8, 6 | 24, 26, 8, 15 | 7 | 35.3 | 24, 11, 12, 6, 9, |
| | | | | | | | | 13 |
| 8 | 2.59 | m | | 24, 7, 12, 9 | 24, 7, 12, 9 | 8 | 30.8 | 24, 9, 13 |
| 9 | 5.36 | m | | 25, 8 | 24, 25, 8 | 9 | 128.0 | 24, 25, 11 |
| — | | | | | | 10 | 132.3 | 25, 11 |
| 11α | 2.02 | dd | 16.9, | 11β, 12 | 11, 12 | 11 | 38.0 | 25, 12, 9, 13 |
| | | | 4.1 | | | | | |
| 11β | 1.75 | m | | 11α, 12 | | | | |
| 12 | 2.13 | m | | 11, 8, 6 | 11, 8, 6 | 12 | 30.7 | 11, 8, 6 |
| 13 | 5.75 | br | 10.2 | 15, 14 | 14 | 13 | 133.4 | 14 |
| | | d | | | | | | |
| 14 | 5.50 | m | | 15, 13 | 15, 13 | 14 | 124.9 | |
| 15 | 2.66 | m | | 26, 16, 6, 17, 14, | 7, 14, 4, 19 | 15 | 45.3 | 26, 16, 6, 14, 13 |
| | | | | 13 | | | | |
| 16 | 2.61 | m | | 26, 15, 17 | 26, 19 | 16 | 32.8 | 26, 6, 18 |
| 17 | 5.41 | m | | 16, 15, 18 | 26, 18 | 17 | 139.1 | 26, 16, 19 |
| 18 | 5.88 | br t | 11.4 | 17, 19 | 17, 21, 19 | 18 | 125.7 | 16, 20, 19 |
| 19 | 6.42 | m | | 21, 20, 18 | 16, 15, 21, 18, | 19 | 123.7 | 17, 21, 20, 18 |
| | | | | | 4 | | | |
| 20 | 5.58 | m | | 21, 19 | | 20 | 131.2 | 22, 21, 18, 19 |
| 21 | 5.55 | m | | 22, 20, 19 | 22, 18, 19 | 21 | 69.8 | 22, 20, 19 |
| 22 | 1.34 | d | 6.7 | 21 | 21 | 22 | 20.5 | 21 |
| 23 | 1.50 | m | | 2 | 2, 4 | 23 | 14.7 | 2 |
| 24 | 0.76 | d | 7.2 | 8 | 7, 8, 6, 9 | 24 | 15.3 | 7 |
| 25 | 1.67 | s | | 9 | 9 | 25 | 23.4 | 11 |
| 26 | 0.95 | d | 6.6 | 16, 15 | 7, 16, 17 | 26 | 16.3 | 16, 17 |
| OH1 | 15.66 | br s | | | | — | | |

### ChB1-2′Epi

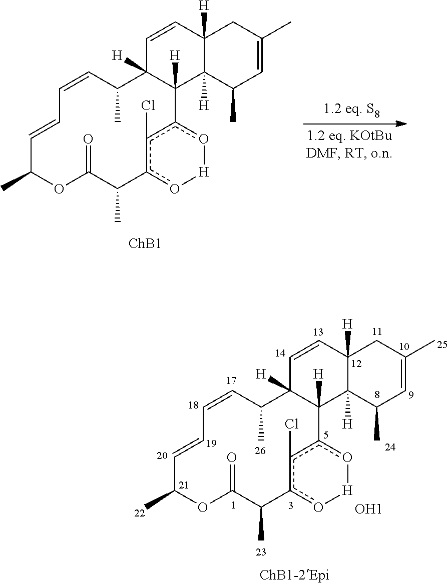

ChB1-2′Epi was obtained from a dehalogenation reaction (see above; 70 mg of a dehalogenation reaction optimization mixture) by semi preparative HPLC. The gradient used was 30% ACN-95% ACN in 30 min. HRMS: C₂₆H₃₄O₄Cl [M+H]⁺: calc.: 445.2140, found: 445.2139; Δ=0.3.

### NMR Table of ChB1-2′Epi

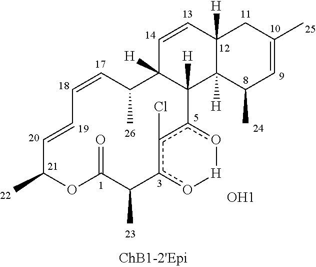

ChB1-2′Epi

| H | δ_{H} | m | J(Hz) | COSY | NOESY | C | δ_{C} | HMBC |
|---|---|---|---|---|---|---|---|---|
| — | | | | | | 1 | 168.3 | 23, 2, 21 |
| 2 | 5.01 | q | 6.8 | 23 | 22, 23 | 2 | 45.7 | 23 |
| — | | | | | | 3 | 196.3 | 23, 2 |
| 4 | | | | | | 4 | 107.0 | 15, 2 |
| — | | | | | | 5 | 204.5 | 15 |
| 6 | 2.79 | br s | | 12, 7, 16, | 12, 7, 16, 15, 17, 14, | 6 | 44.4 | 12, 7, 16, 15, 14, |
| | | | | 15, 14 | 13, 19 | | | 13 |
| 7 | 2.22 | m | | 12, 8, 6, 15 | 24, 26, 8, 6, 15, 9, | 7 | 31.0 | 24, 11, 12, 8, 15, 9, |
| | | | | | 14, 13 | | | 14, 13 |
| 8 | 2.52 | br s | | 24, 25, 12, | 24, 7, 9 | 8 | 30.9 | 24, 9 |
| | | | | 7, 9 | | | | |
| 9 | 5.35 | m | | 24, 25, 11, 8 | 24, 25, 11, 7, 8 | 9 | 128.2 | 24, 25, 11, 7, 8 |
| — | | | | | | 10 | 132.5 | 25, 11, 8 |
| 11α | 1.76 | m | | 11β, 12, 9 | 26, 25, 11β, 12, 13 | 11 | 38.2 | 25, 12, 9, 13 |
| 11β | 2.04 | m | | 11α, 12, 9 | 25, 11α, 12, 9, 13 | | | |
| 12 | 2.20 | m | | 11, 7, 8, 6 | 24, 11, 6, 15, 13 | 12 | 36.1 | 11, 8, 6, 15, 9, 13 |
| 13 | 5.78 | br d | 10.1 | 14 | 26, 25, 11, 12, 7, 6, | 13 | 133.6 | 11, 12, 6, 15, 14 |
| | | | | | 14 | | | |
| 14 | 5.53 | m | | 6, 13 | 7, 6, 13, 18 | 14 | 124.1 | 12, 16, 6, 13 |
| 15 | 3.65 | br | 11.1, | 7, 6 | 24, 12, 7, 6 | 15 | 48.1 | 7, 6, 14 |
| | | dd | 6.4 | | | | | |
| 16 | 2.47 | m | | 26, 6, 17 | 26, 6, 17, 20, 18, 19 | 16 | 33.0 | 26, 6, 15, 17, 18 |
| 17 | 5.39 | m | | 16, 18 | 26, 16, 6, 20, 18 | 17 | 138.9 | 26, 16, 6, 19 |
| 18 | 5.86 | br t | 10.9 | 17, 19 | 16, 17, 14, 20, 19 | 18 | 125.4 | 16, 21, 20, 19 |
| 19 | 6.13 | m | | 20, 18 | 22, 16, 6, 20, 18 | 19 | 123.6 | 17, 21, 20, 18 |
| 20 | 5.56 | m | | 22, 19 | 26, 22, 16, 17, 18, | 20 | 131.5 | 22, 17, 21, 18, 19 |
| | | | | | 19 | | | |
| 21 | 5.47 | m | | 22 | 22 | 21 | 70.3 | 22, 20, 19 |
| 22 | 1.33 | d | 6.7 | 21, 20 | 2, 21, 20, 19 | 22 | 20.9 | 21, 20 |
| 23 | 1.50 | d | 6.9 | 2 | 2 | 23 | 13.1 | 2 |
| 24 | 0.79 | d | 7.0 | 8, 9 | 12, 7, 8, 15, 9 | 24 | 16.2 | 7,9 |
| 25 | 1.67 | s | | 8, 9 | 11, 9, 13 | 25 | 23.5 | 9 |
| 26 | 0.95 | d | 6.6 | 16 | 11, 7, 16, 17, 20, 13 | 26 | 16.7 | 16, 17 |

### Dehalogenil-Epo1/Dehalogenil-Epo2

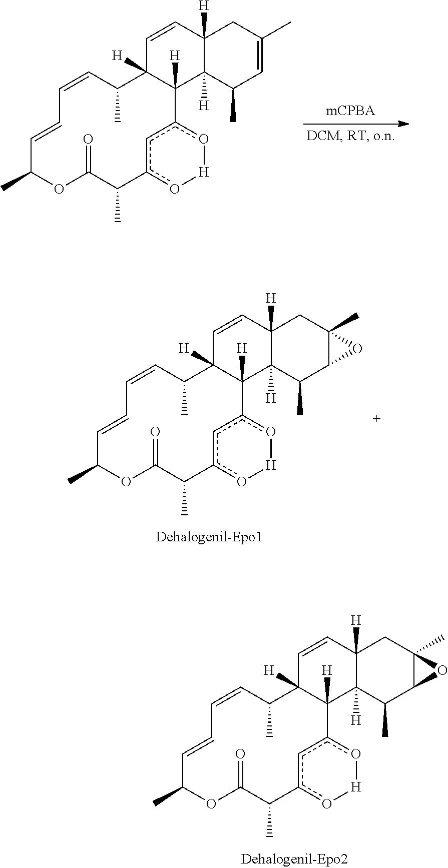

Dehalogenil (30.0 mg; 73 μmol) and 1.4 eq. mCPBA (17.7 mg; 102 μmol were dissolved in 3.5 ml DCM. The reaction was stirred overnight at room temperature. Upon completion, the mixture was quenched with Na₂SO₄ and extracted three times with 5 ml DCM. The combined organic phases were washed with water, dried over MgSO₄ and the solvent was evaporated. The reaction was purified by preparative HPLC with an eluent gradient of Water:MeCN 5:95-0:100. Yield: 6 mg of GJE-326-1, 14 μmol, 19%, pale yellow powder and 15 mg of GJE-326-2, 35 μmol, 48%, pale yellow powder.

HRMS GJE-326-1: C₂₆H₃₃O₅ [M−H]⁻: calc.: 425.23335, found: 425.23346; Δ=0.26 ppm.

HRMS GJE-326-3: C₂₆H₃₃O₅⁻[M−H]⁻: calc.: 425.23335, found: 425.23328; Δ=0.16 ppm.

### NMR Table of Dehalogenil-Epo1

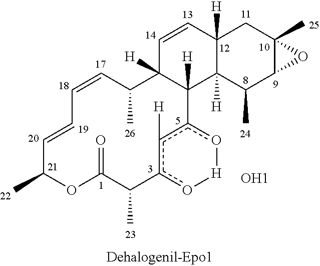

Dehalogenil-Epo1

| H | δ_{H} | m | J(Hz) | COSY | C | δ_{C} | HMBC |
|---|---|---|---|---|---|---|---|
| — | | | | | 1 | 168.9 | 23, 2, 21 |
| 2 | 3.51 | q | 6.9 | | 2 | 49.1 | 23, 4 |
| — | | | | | 3 | 190.7 | 23, 2 |
| 4 | 5.96 | s | | | 4 | 102.6 | 6, 2 |
| — | | | | | 5 | 194.2 | 6 |
| 6 | 2.62 | dd | 12.0, 6.6 | 7, 12 | 6 | 48.6 | 8 |
| 7 | 1.95 | m | | 12, 6, 8 | 7 | 29.0 | 24, 11, 15, 6, 9, 13 |
| 8 | 2.60 | m | | 24, 9 | 8 | 32.8 | 24, 9, 12, 7 |
| 9 | 2.79 | br s | | 8 | 9 | 65.6 | 7 |
| — | | | | | 10 | 57.5 | 25, 11 |
| 11α | 1.60 | m | | 11β, 12 | 11 | 36.5 | 25, 9, 12, 13 |
| 11β | 1.95 | m | | 11α | | | |
| 12 | 2.09 | m | | 11 | 12 | 30.2 | 11, 8, 6, 13 |
| 13 | 5.54 | m | | 14 | 13 | 132.4 | 15 |
| 14 | 5.50 | m | | 16, 15, 13 | 14 | 125.0 | 15, 16 |
| 15 | 2.61 | m | | 14 | 15 | 46.1 | 26, 16, 6, 14, 13 |
| 16 | 2.67 | m | | 26, 17, 14 | 16 | 29.8 | 26, 15, 17, 18 |
| 17 | 5.36 | t | 9.4 | 16, 18 | 17 | 139.0 | 26, 16, 15, 19 |
| 18 | 5.86 | t | 10.9 | 17, 19 | 18 | 126.0 | 16, 20, 19 |
| 19 | 6.43 | br t | 12.7 | 20, 18 | 19 | 123.8 | 17, 20, 21, 18 |
| 20 | 5.53 | m | | 19 | 20 | 131.7 | 22, 21, 18, 19 |
| 21 | 5.56 | m | | 22 | 21 | 69.9 | 22, 20, 19 |
| 22 | 1.33 | d | 6.8 | 21 | 22 | 21.0 | 21 |
| 23 | 1.41 | d | 6.9 | 2 | 23 | 11.9 | 2 |
| 24 | 0.87 | d | 7.1 | 8 | 24 | 10.9 | 8, 7 |
| 25 | 1.34 | s | | 9 | 25 | 24.9 | 11, 9 |
| 26 | 0.92 | d | 6.5 | 16 | 26 | 16.6 | 16, 17 |
| OH1 | 15.49 | br s | | | — | | |

### NMR Table of Dehalogenil-Epo2

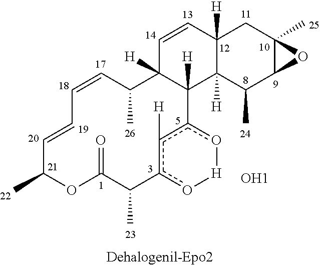

Dehalogenil-Epo2

| H | δ_{H} | m | J(Hz) | COSY | C | δ_{C} | HMBC |
|---|---|---|---|---|---|---|---|
| — | | | | | 1 | 168.9 | 23, 2, 21 |
| 2 | 3.51 | q | 6.9 | | 2 | 48.6 | 23, 4 |
| — | | | | | 3 | 189.2 | 23, 2 |
| 4 | 5.97 | s | | | 4 | 102.3 | 6, 2 |
| — | | | | | 5 | 196.3 | 6 |
| 6 | 2.62 | dd | 12.0, 6.6 | 7, 12 | 6 | 48.1 | 8 |
| 7 | 1.89 | m | | 12, 6, 8 | 7 | 35.6 | 24, 11, 15, 6, 9, 13 |
| 8 | 2.58 | m | | 24, 9 | 8 | 32.8 | 24, 9, 12, 7 |
| 9 | 3.03 | d | 5.4 | 8 | 9 | 64.9 | 7 |
| — | | | | | 10 | 59.4 | 25, 11 |
| 11α | 1.40 | m | | 11β, 12 | 11 | 38.9 | 25, 9, 12, 13 |
| 11β | 2.09 | br dd | 14.3, 3.7 | 11α | | | |
| 12 | 2.03 | m | | 11 | 12 | 28.5 | 11, 8, 6, 13 |
| 13 | 5.61 | br d | 10.0 | 14 | 13 | 132.9 | 15 |
| 14 | 5.49 | m | | 16, 15, 13 | 14 | 124.5 | 15, 16 |
| 15 | 2.61 | m | | 14 | 15 | 45.6 | 26, 16, 6, 14, 13 |
| 16 | 2.62 | m | | 26, 17, 14 | 16 | 32.8 | 26, 15, 17, 18 |
| 17 | 5.35 | t | 9.4 | 16, 18 | 17 | 138.9 | 26, 16, 15, 19 |
| 18 | 5.86 | t | 10.9 | 17, 19 | 18 | 126.1 | 16, 20, 19 |
| 19 | 6.41 | br t | 12.7 | 20, 18 | 19 | 123.7 | 17, 20, 21, 18 |
| 20 | 5.55 | m | | 19 | 20 | 131.6 | 22, 21, 18, 19 |
| 21 | 5.57 | m | | 22 | 21 | 69.9 | 22, 20, 19 |
| 22 | 1.33 | d | 6.8 | 21 | 22 | 20.9 | 21 |
| 23 | 1.42 | d | 6.9 | 2 | 23 | 11.9 | 2 |
| 24 | 0.86 | d | 7.1 | 8 | 24 | 10.3 | 8, 7 |
| 25 | 1.35 | s | | 9 | 25 | 23.0 | 11, 9 |
| 26 | 0.92 | d | 6.5 | 16 | 26 | 16.5 | 16, 17 |
| OH1 | 15.59 | br s | | | — | | |

### Thiotonil

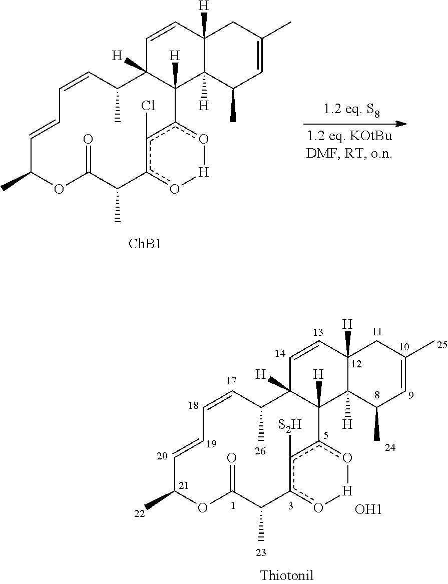

Thiotonil was obtained as a minor component from a dehalogenation reaction (see above; 70 mg of dehalogenation reaction optimization mixture) by semi preparative HPLC. The gradient used was 30% ACN-95% ACN in 30 min. Yield: 3.1 mg; 6.5 μmol, white solid. HRMS: C₂₆H₃₅O₄S₂ [M+H]⁺: calc.: 473.1815, found: 473.1812; Δ=0.6 ppm.

### NMR Table of Thiotonil

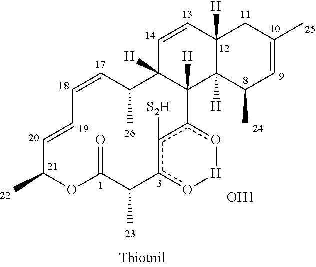

Thiotonil

| H | δ_{H} | m | J(Hz) | COSY | C | δ_{C} | HMBC |
|---|---|---|---|---|---|---|---|
| — | | | | | 1 | 198.9 | |
| 2 | 5.35 | m | | 23 | 2 | 72.5 | 23 |
| — | | | | | 3 | 167.1 | 2 |
| 4 | | | | | 4 | 108.4 | 6 |
| — | | | | | 5 | 177.8 | 7, 6 |
| 6 | 2.73 | dd | 11.2, 6.9 | 12, 7, 15 | 6 | 49.3 | 12, 7 |
| 7 | 2.22 | m | | 12, 16, 8, 6 | 7 | 30.8 | 12, 6, 13 |
| 8 | 2.52 | m | | 25, 24, 11, 7, 9 | 8 | 31.3 | 26, 7, 6, 9 |
| 9 | 5.39 | br d | 4.1 | 25, 24, 11, 16, 8 | 9 | 127.9 | 25, 11, 16, 8 |
| — | | | | | 10 | 132.6 | 25, 11, 16, 8 |
| 11α | 2.04 | br dd | 16.7, 3.9 | 25, 11β, 12, 9 | 11 | 37.9 | 25, 9, 14 |
| 11β | 1.77 | m | | 25, 11α, 12, 16, 8, 9 | | | |
| 12 | 2.17 | m | | 11, 7, 6, 13, 14 | 12 | 36.0 | 11, 6, 9, 14 |
| 13 | 5.53 | m | | 12, 15, 14 | 13 | 124.1 | 12, 15, 14 |
| 14 | 5.77 | br d | 10.2 | 12, 15, 13 | 14 | 133.1 | 12, 15, 6, 13 |
| 15 | 2.59 | m | | 26, 16, 8, 6, 13, 14 | 15 | 43.8 | 6, 13, 14 |
| 16 | 2.52 | m | | 26, 15 | 16 | 31.3 | 26, 7, 6, 9 |
| 17 | 5.61 | br d | 15.4 | 18 | 17 | 130.5 | 20, 19, 18 |
| 18 | 6.54 | m | | 17, 19 | 18 | 123.4 | 20, 17, 19 |
| 19 | 5.92 | t | 10.8 | 21, 20, 18 | 19 | 125.9 | 22, 21, 17, 18 |
| 20 | 5.43 | br t | 9.2 | 22, 21, 19 | 20 | 139.1 | 22, 21, 18 |
| 21 | 2.80 | quin | 7.4 | 22, 20, 19 | 21 | 32.9 | 22, 20, 19 |
| 22 | 1.01 | d | 6.6 | 21, 20 | 22 | 17.0 | 21,20 |
| 23 | 1.44 | d | 6.3 | 2 | 23 | 19.8 | 2 |
| 24 | 1.73 | s | | 16, 8, 9 | 24 | 19.2 | |
| 25 | 1.68 | s | | 11, 16, 8, 9 | 25 | 23.4 | 11, 9 |
| 26 | 0.84 | d | 7.0 | 16, 8, 15 | 26 | 15.2 | |
| SH1 | 1.59 | br s | | | — | | |
| OH1 | 13.80 | s | | | — | | |

### ChA-9′Cl

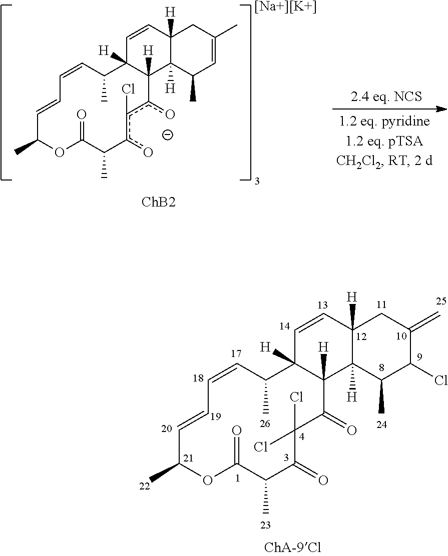

ChB2 (10.0 mg; 23 μmol) and 2.4 eq. N-chlorosuccinimide (NCS, 7.2 mg; 54 μmol) were dissolved in 1 mL dry CH₂Cl₂ followed by addition of 1.2 eq. pyridine (2.1 mg; 27 μmol; 2.1 μl; one drop). The reaction was stirred for two days at room temperature. Afterwards, 1.2 eq. pTSA*H₂O (5.6 mg; 27 μmol) was added and the solution was stirred over night at room temperature. The mixture was extracted three times with 10 mL CH₂Cl₂ from 10 mL water. The combined organic phases were dried over MgSO₄ and the solvent was evaporated. The reaction was purified by flash chromatography over silica preequilibrated in hexane with an eluent gradient of Hex:CH₂Cl₂ 5:1-5:1.25-5.1.5-5:2-1:1-3:2-CH₂Cl₂. Yield: 0.5 mg; 1 μmol; 4%, white solid. R_{f} (Silica, CH₂Cl₂:Hex 3:2): 0.38. Diastereomeric mixture. HRMS: C₂₆H₃₂Cl₃O₄ [M+H]⁺: calc.: 513.13607, found: 513.13607; Δ=0.23 ppm.

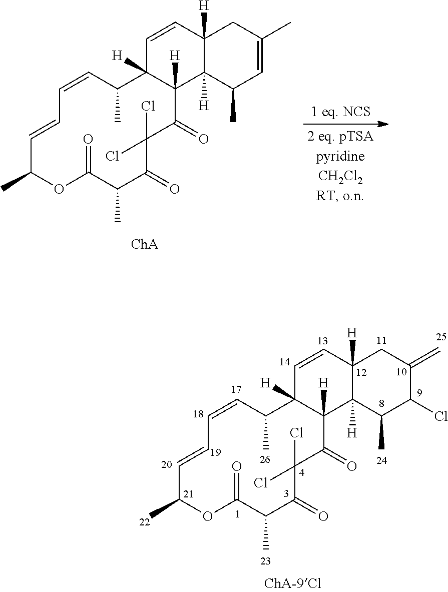

ChA (10.0 mg; 21 μmol), 1 eq. N-chlorosuccinimide (2.8 mg; 21 μmol) and 2.0 eq. pTSA (8.6 mg; 42 μmol) were dissolved in 1 mL dry CH₂Cl₂ and three drops of dry pyridine was added. The reaction was stirred at RT o.n. The reaction was poured into 10 mL water and was extracted three times with 10 mL CH₂Cl₂. The organic phase was dried over MgSO₄ and the solvent evaporated. The mixture was extracted three times with 10 mL CH₂Cl₂ from 10 mL water. The combined organic phases were dried over MgSO₄ and the solvent was evaporated. The reaction was purified by flash chromatography over silica preequilibrated in hexane with an eluent gradient of Hex:CH₂Cl₂ 5:1-5:1.25-5.1.5-5:2-1:1-3:2-CH₂Cl₂. Yield: 7.1 mg; 13.9 μmol; 33%, white solid. Diastereomeric mixture.

### NMR Table of ChA-9′CI-Diastereomer 1

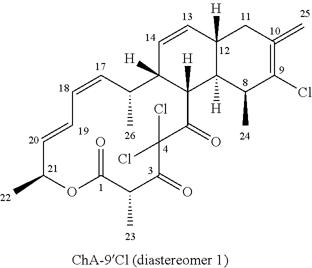

ChA-9′Cl (diastereomer 1)

| H | δ_{H} | m | J(Hz) | COSY | NOESY | C | δ_{C} | HMBC |
|---|---|---|---|---|---|---|---|---|
| — | | | | | | 1 | 168.0 | 23, 2 |
| 2 | 4.53 | q | 7.0 | 23 | 23 | 2 | 47.2 | 23 |
| — | | | | | | 3 | 192.0 | 23, 2 |
| — | | | | | | 4 | | |
| — | | | | | | 5 | 196.0 | 6 |
| 6 | 3.85 | dd | 11.1, 7.0 | 7, 15 | 24, 12, 7, 15, 9 | 6 | 50.4 | 13 |
| 7 | 2.16 | m | | 12, 8, 6 | 24, 26, 12, 8, 6, | 7 | 41.2 | 24, 6 |
| | | | | | 9 | | | |
| 8 | 2.45 | ddd | 7.1, 4.4, | 24, 7, 9 | 24, 7, 11, 9 | 8 | 37.6 | 24, 9 |
| | | | 2.9 | | | | | |
| 9 | 4.66 | m | | 11, 8, 25 | 24, 11, 12, 7, 8, | 9 | 66.6 | 24, 8, 11, 25 |
| | | | | | 6 | | | |
| — | | | | | | 10 | 142.4 | 11, 8, 11, 9, 25 |
| 11α | 1.88 | br t | 12.4 | 12, 11β, 9, 25 | 12, 11β, 9, 14 | | 41.5 | 25, 14 |
| 11β | 2.58 | dd | 13.3, 3.5 | 11α, 12, 25 | 11α, 12, 8, 25, | 11 | | |
| | | | | | 14 | | | |
| 12 | 2.10 | m | | 11, 7, 11, 13, | 24, 11, 7, 6, 9, | 12 | 34.2 | 11, 6, 13, 14 |
| | | | | 14 | 14 | | | |
| 13 | 5.51 | m | | 12, 15, 14 | 15, 17, 14 | 13 | 124.8 | 16, 15 |
| 14 | 5.66 | br d | 10.1 | 12, 15, 13 | 11, 12, 15, 13 | 14 | 132.2 | 11, 13 |
| 15 | 3.01 | m | | 16, 6, 13, 14 | 16, 6, 13, 14, 19 | 15 | 43.1 | 26, 6, 13, 14 |
| 16 | 2.76 | m | | 26, 15, 17 | 26, 15, 17, 18, | 16 | 33.5 | 26, 6, 17, 18 |
| | | | | | 19 | | | |
| 17 | 5.32 | m | | 16, 18, 19 | 26, 16, 13, 20, | 17 | 138.7 | 26, 19 |
| | | | | | 18 | | | |
| 18 | 5.89 | t | 10.9 | 17, 19 | 16, 17, 20, 19 | 18 | 125.9 | 26, 17, 20 |
| 19 | 6.05 | m | | 17, 20, 21, 18 | 16, 15, 20, 18 | 19 | 123.9 | 22, 17, 20, 21, |
| | | | | | | | | 18 |
| 20 | 5.53 | m | | 22, 21, 19 | 26, 22, 17, 18, | 20 | 130.7 | 22, 21, 18 |
| | | | | | 19 | | | |
| 21 | 5.59 | m | | 22, 20, 19 | 22 | 21 | 70.5 | 22, 20, 19 |
| 22 | 1.32 | d | 6.6 | 20, 21 | 20, 21 | 22 | 21.1 | 20, 21 |
| 23 | 1.66 | d | 7.0 | 2 | 2 | 23 | 17.0 | 2 |
| 24 | 0.82 | d | 7.0 | 8 | 12, 7, 8, 6, 9, 25 | 24 | 7.1 | 8, 9 |
| 25′ | 5.22 | d | 1.5 | 11, 9, 25′ | 24, 25″ | 25 | 111.8 | 11, 9 |
| 25″ | 5.04 | d | 1.4 | 11, 9, 25″ | 24, 11, 25′ | | | |
| 26 | 0.98 | d | 6.4 | 16 | 16, 17, 20 | 26 | 15.7 | 17 |

### NMR Table of ChA-9′CI-Diastereomer 2

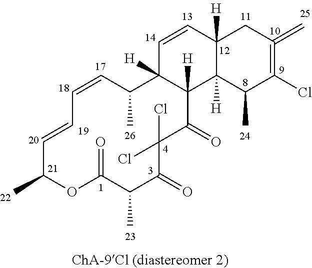

ChA-9′Cl (diastereomer 2)

| H | δ_{H} | m | J(Hz) | COSY | C | δ_{C} | HMBC |
|---|---|---|---|---|---|---|---|
| — | | | | | 1 | 167.9 | 23, 2 |
| 2 | 4.54 | m | m | 23 | 2 | 47.0 | 23 |
| — | | | | | 3 | 191.8 | 23, 2 |
| — | | | | | 4 | 80.9 | |
| — | | | | | 5 | 195.6 | 6 |
| 6 | 3.78 | dd | 11.9, 6.9 | 7, 15 | 6 | 49.8 | 13 |
| 7 | 2.33 | m | | 12, 8, 6 | 7 | 36.1 | 24, 6 |
| 8 | 2.44 | m | | 24, 7, 9 | 8 | 37.1 | 24, 9 |
| 9 | 4.43 | d | 2.0 | 11, 8, 25 | 9 | 67.8 | 24, 8, 11, 25 |
| — | | | | | 10 | 143.6 | 11, 8, 9, 25 |
| 11α | 2.58 | m | | 12, 11β, 9, 25 | 11 | 41.3 | 25, 14 |
| 11β | 1.90 | m | | 11α, 12, 25 | | | |
| 12 | 2.12 | m | | 11, 7, 13, 14 | 12 | 34.2 | 11, 6, 13, 14 |
| 13 | 5.52 | m | | 12, 15, 14 | 13 | 124.5 | 16, 15 |
| 14 | 5.67 | br d | 9.9 | 12, 15, 13 | 14 | 132.7 | 11, 13 |
| 15 | 3.04 | m | | 16, 6, 13, 14 | 15 | 43.2 | 26, 6, 13, 14 |
| 16 | 2.79 | m | | 26, 15, 17 | 16 | 33.4 | 26, 6, 17, 18 |
| 17 | 5.35 | m | | 16, 18, 19 | 17 | 138.9 | 26, 19 |
| 18 | 5.91 | t | 10.9 | 17, 19 | 18 | 125.6 | 26, 17, 20 |
| 19 | 6.07 | m | | 17, 20, 21, 18 | 19 | 123.9 | 22, 17, 20, 21, 18 |
| 20 | 5.55 | m | | 22, 21, 19 | 20 | 130.4 | 22, 21, 18 |
| 21 | 5.60 | m | | 22, 20, 19 | 21 | 70.4 | 22, 20, 19 |
| 22 | 1.34 | d | 6.7 | 20, 21 | 22 | 20.9 | 20, 21 |
| 23 | 1.66 | dd | 7.0, 4.0 | 2 | 23 | 16.8 | 2 |
| 24 | 0.83 | d | 7.3 | 8 | 24 | 12.3 | 8, 9 |
| 25 | 4.99 | dt | 18.5, 1.5 | 11, 9, 25 | 25 | 114.0 | 11, 9 |
| 26 | 1.03 | d | 6.4 | 16 | 26 | 15.5 | 17 |

### Biological Data

### MIC Testing:

Minimal inhibitory concentration (MIC) of the compounds in the present invention was determined using broth microdilution performed according to EUCAST guidelines (ISO20776-1:2019). S. aureus ATCC29213 and E. faecalis ATCC29212 served as quality control strains.

Drug Sensitivity Assays Against P. falciparum:

Drug sensitivity assays of asexual plasmodium stages were carried out as described before (doi: 10.1128/AAC.49.8.3575-3577.2005). Briefly, the drugs were distributed in a 3-fold serial dilution in 96-well plates. Synchronized ring-stage parasites were diluted to a parasitemia of 0.05% with O Rh+ erythrocytes incomplete culture medium and seeded at a hematocrit of 1.5% in a total volume of 225 μL per well. Thereafter, the plates were incubated at 5% CO₂, 5% O₂, at 37° C. for 72 h, then they were frozen and thawed three times. The growth inhibition of P. falciparum was measured through an enzyme-linked immunosorbent assay (ELISA) for histidine-rich protein 2 (HRP2) using a microplate reader.

TABLE 1
MIC against S. aureus Newman, S. aureus N315 (methicillin-resistant,
MRSA), E. faecalis ATCC-29212, E. faecium DSM-20477, E. faecium DSM-
17050 (vancomycin-resistant, VRE), S. epidermidis DSM-28765. Half-
inhibitory concentrations (IC₅₀) are shown as mean ± SD
(n = 2) and were determined for drug-sensitive P. falciparum
3D7 (chloroquine IC₅₀ = 7.4 ± 2.6 nM) and chloroquine-
resistant P. falciparum Dd2 (chloroquine IC₅₀ =
226 ± 76 nM).

| | MIC [μg/mL] | | | | | | IC₅₀ [MM] | |
|---|---|---|---|---|---|---|---|---|
| | S. | | E. | E. | | S. | P. falcip. | P. falcip. |
| Compound | aureus | MRSA | faecalis | faecium | VRE | epiderm. | 3D7 | Dd2 |
| ChA | 0.0125 | 0.05 | 0.025 | 0.0125 | 0.025 | 0.05 | 0.014 ± | 0.016 ± |
| | | | | | | | 0.005 | 0.004 |
| ChB1 | 0.2 | 0.8 | 0.2 | 0.1 | 0.1 | 0.4 | 0.04 ± | 0.045 ± |
| | | | | | | | 0.004 | 0.003 |
| ChB2 | 3.2 | >3.2 | 1.6 | 0.4 | 0.8 | 3.2 | 0.091 ± | 0.063 ± |
| | | | | | | | 0.010 | 0.008 |
| ChB1-Epo2 | 0.05 | 0.05 | 0.1 | 0.05 | 0.05 | 0.1 | 0.220 ± | 0.372 ± |
| | | | | | | | 0.029 | 0.202 |
| dehalogenil- | 0.8 | 0.8 | 1.6 | 0.8 | 0.8 | 3.2 | 0.408 ± | 0.385 ± |
| 2′DM | | | | | | | 0.173 | 0.130 |
| Dehalogenil- | 3.2 | 3.2 | 3.2 | 3.2 | 3.2 | 3.2 | 2.03 ± | 2.05 ± |
| Epo1 | | | | | | | 0.182 | 0.705 |
| Dehalogenil- | 0.4 | 0.2 | 1.6 | 0.8 | 0.4 | 0.8 | 0.198 ± | 0.142 ± |
| Epo2 | | | | | | | 0.016 | 0.009 |
| ChB1-2′DM | 0.4 | 0.4 | 0.4 | 0.4 | 0.4 | 1.6 | — | — |
| ChA-4′F | 0.8 | 1.6 | 1.6 | 0.8 | 1.6 | >6.4 | 0.572 ± | 0.415 ± |
| | | | | | | | 0.021 | 0.007 |
| ChA-4′Me | 1.6 | 1.6 | 1.6 | 0.8 | 0.8 | >1.6 | >0.242 | >0.242 |
| ChB1-2′Me | 0.4 | 0.8 | 0.8 | 0.2 | 0.4 | 0.8 | 0.108 ± | 0.071 ± |
| | | | | | | | 0.003 | 0.003 |
| dehalogenil | 0.05 | 0.05 | 0.1 | 0.025 | 0.1 | 0.2 | 0.005 ± | 0.0039 ± |
| | | | | | | | 0.002 | 0.0002 |
| dehalogenil- | 0.2 | 0.1 | 0.2 | 0.05 | 0.1 | 0.4 | 0.028 ± | 0.060 ± |
| 2′Epi | | | | | | | 0.000 | 0.044 |
| thiotonil | 0.4 | 0.4 | 0.2 | 0.025 | 0.025 | 0.4 | 0.056 ± | 0.065 ± |
| | | | | | | | 0.002 | 0.002 |
| ChA-9′Cl | 0.4 | 0.4 | 0.4 | 0.2 | 0.2 | 0.4 | 3.328 ± | 1.886 ± |
| | | | | | | | 1.749 | 0.346 |

### Activity Against Mature Gametocytes (Stage IV-V) Assay

Activity against gametocytes was determined using an in vitro bioluminescence assay as described previously (doi.org/10.1371/journal.pone.0035019).

TABLE 2
Activity of chlorotonil derivatives against
mature gametocytes (stages IV and V).

| | Mature gametocytes |
|---|---|
| Compound | (stages IV and V) IC₅₀ [nM] |
| ChA | 9.5 ± 3 |
| ChB1-Epo2 | 347.5 ± 18.6 |
| Dehalogenil-2′DM | 499.1 ± 86.8 |
| dehalogenil | 5.8 ± 0 |
| dehalogenil-2′Epi | 25.2 ± 1.63 |
| thiotonil | 67.4 ± 8.60 |

### MIC in Presence of Fetal Bovine Serum (FBS)

MIC of selected molecules against S. aureus Newman was determined in the presence of fetal bovine serum following the protocol described above, where FBS was added at different concentrations to the assay medium Both ChA and Dehalogenil remained active above 20% FBS whereas ChB1-Epo2 already lost 100-fold activity at 5% FBS.

TABLE 3
MIC [μg/ml] against S. aureus Newman
in presence of varying percentage of FBS.

| Medium | ChA | ChB1-Epo2 | dehalogenil |
|---|---|---|---|
| MHB2 | 0.025 | 0.03 | 0.0625 |
| MHB2 + 5% FBS | 0.8 | 4 | 2 |
| MHB2 + 10% FBS | 0.8 | 4 | 4 |
| MHB2 + 20% FBS | 1.6 | 8 | 4 |
| MHB2 + 30% FBS | 3.2 | >8 | 8 |
| MHB2 + 40% FBS | 3.2 | >8 | 8 |
| MHB2 + 50% FBS | 3.2 | >8 | >8 |

### MIC 50/90 Profiling

Activity of dehalogenil against multidrug-resistant clinical isolates of S. aureus (n=100) and Enterococcus spp. (n=98) was confirmed demonstrating resistance-breaking properties. Compared to the standard of care (linezolid, daptomycin and tigecyclin), dehalogenil MIC₉₀ was lowered in both pathogens by 20-40-fold with unimodal distribution indicating no pre-existing resistances.

TABLE 4
MIC distribution of dehalogenil and three clinically approved
antibiotics in clinical isolates of S. aureus (n = 100).

| | S. aureus (n = 100) | | | E. faecium (n = 98) | | |
|---|---|---|---|---|---|---|
| | MIC range | MIC₅₀ | MIC₉₀ | MIC range | MIC₅₀ | MIC₉₀ |
| | [μg/ml] | [μg/ml] | [μg/ml] | [μg/ml] | [μg/ml] | [μg/ml] |
| dehalogenil | 0.025-0.4 | 0.1 | 0.2 | 0.00625-0.2 | 0.025 | 0.1 |
| linezolid | 2->64 | 4 | 8 | 0.5-64 | 4 | 4 |
| daptomycin | 0.25->64 | 0.5 | 4 | 0.5-4 | 2 | 4 |
| tigecyclin | 1-16 | 4 | 8 | 0.5-16 | 2 | 2 |

### Maximum Tolerated Concentration (MTC) in Zebrafish Larvae Assays

For toxicity screening, zebrafish larvae were exposed three days post fertilization and monitored for 48 h until five days post fertilization. All malformations and pathophysiological phenotypes were noted and used to determine the inhibitory concentration (IC₅₀) of the respective derivative against zebrafish larvae. The selectivity index (SI) was then calculated using the IC₅₀ and the MIC of either S. aureus or P. falciparum 3D7. Derivatives of the current invention were found to be less toxic compared to ChA and ChB1-Epo2, significantly improving the safety margin notably without losing antimicrobial activity.

TABLE 5
MTC in zebrafish larvae and selectivity indices against
S. aureus Newman and P. falciparum 3D7.

| | |
|---|---|
| IC₅₀ | Selectivity Index |

| Derivative | [μM] | S. aureus | P. falc 3D7 |
|---|---|---|---|
| ChA | 1.09 | 41.7 | 77.8 |
| ChB1-Epo2 | 1.14 | 10.5 | 5.2 |
| dehalogenil | 27.4 | 224.8 | 5479.6 |
| dehalogenil-2′DM | 27.03 | 13.4 | 66.2 |
| dehalogenil-2′Epi | 38.46 | 78.9 | 1373.7 |
| ChB2-K | 8.25 | 39.9 | 257.7 |
| thiotonil | >50 | >62.5 | >892.9 |
| ChB1-2′DM | 33.24 | 35.8 | |
| ChB1-2′Me | 12.31 | 14.1 | 114.0 |
| Dehalogenil-Epo1 | 9.85 | 1.31 | 4.9 |
| Dehalogenil-Epo2 | 1.22 | 1.30 | 6.2 |

### ADME Profiling

Complementing the toxicity profiling, ADME properties of the lead dehalogenil were determined. This had not been possible for the first generation frontrunner ChB1-Epo2 or the natural product ChA because of their poor limit of detection. Overall, dehalogenil showed a favourable ADME profile. Solubility more than doubled at pH 7.4 when compared to ChB1-Epo2 (kinetic solubility, 16.2±4.9 μM) and increased more than 500-fold compared to the natural product ChA. Across species (mouse, rat, human), metabolic stability is high while plasma stability is moderate. Besides, no hemolysis and no inhibition of the metabolically important enzyme CYP3A4 was observed by both dehalogenil and ChA. Plasma protein binding (PPB) is >99.4% in all species.

TABLE 6
ADME profiling of dehalogenil.

| | semi thermodynamic solubility [μM] | | |
|---|---|---|---|
| | pH 5.0 | pH 7.4 | pH 9.0 |
| | 34.9 ± 2.7 | 41.4 ± 10.3 | 16.5 ± 8.7 |
| | mouse | rat | human |
| microsomal clearance | 6.4 | 14 | 6.4 |
| [μL/min/mg protein] | | | |
| plasma stability t_{1/2} [h] | 1.5 | 1.2 | 1.4 |
| plasma protein binding [%] | >99.8 | >99.4 | >99.6 |

| | |
|---|---|
| CYP3A4 inhibition | 16.4 μM |
| haemolysis of human blood | 1.40% |
| cells @ 20 μM | |

### In Vivo Pharmacokinetics (PK)

Peroral administration was compared between dehalogenil and ChB1-Epo2. After oral administration in soy oil, ca. four-fold higher levels were found for cₘₐₓ and ca. six-fold higher AUC_{0-tz} of dehalogenil compared to ChB1-Epo2. Clearance was slow (t_{1/2z} 5.5 and 10 h for 10 and 50 mg/kg, respectively) but considerably higher than for ChB1-Epo2.

TABLE 7
PK parameters after peroral administration of dehalogenil
and ChB1-Epo2 in mice given as mean (n = 3).

| | dehalogenil | | ChB1-Epo2 | |
|---|---|---|---|---|
| | 10 mg/kg, | 50 mg/kg, | 10 mg/kg, | 50 mg/kg, |
| Parameters | p.o. | p.o. | p.o. | p.o. |
| cₘₐₓ [ng/mL] | 261 | 579 | 75.3 | 147.7 |
| tₘₐₓ [h] | 3 | 3 | 3 | 3 |
| t_{z} [h] | 24 | 24 | 18 | 24 |
| t_{1/2z} [h] | 5.5 | 10.2 | 31.9 | n.c. |
| AUC_{0-tz} [ng*h/mL] | 3022 | 8359 | 603 | 1409 |

According to the current invention, intravenous injection in vivo was possible for the first time using a vehicle consisting of 50% ClearSol™ in phosphate buffered saline (PBS) pH 7.4. Based on this, the bioavailability of dehalogenil after peroral administration was determined to be 9.5%.

TABLE 8
Peroral bioavailability of dehalogenil
in mice given as mean (n = 3).

| Parameters^{[a]} | 10 mg/kg po | 7.5 mg/kg iv |
|---|---|---|
| C₀ [ng/mL] | — | 872 |
| cₘₐₓ [ng/mL] | 101 | — |
| tₘₐₓ [h] | 3 | — |
| t_{z} [h] | 24 | 24 |
| t_{1/2z} [h] | 6.7 | 10.8 |
| AUC_{0-tz} [ng*h/mL] | 1400 | 9575 |
| AUC (0-inf) [ng*h/mL] | 1537 | 12120 |
| Bioavailability [%] | 9.5 | 100 |

Dehalogenil was further applied subcutaneously at 5, 10 and 25 mg/kg in 50% ClearSol™/PBS pH 7.4. The determined levels indicate suitable dose response after subcutaneous application with the 25 mg/kg dose being comparable to 7.5 mg/kg dehalogenil applied intravenously.

TABLE 9
PK parameters (n = 3) of dehalogenil
in mice after subcutaneous application.

| Parameters | 5 mg/kg sc | 10 mg/kg sc | 25 mg/kg sc |
|---|---|---|---|
| Cₘₐₓ [ng/mL] | 125.0 | 488.0 | 1195 |
| tₘₐₓ [h] | 7.0 | 3.0 | 3.0 |
| C_{z} [ng/mL] | 109.0 | 257.0 | 595 |
| t_{z} [h] | 24.0 | 24.0 | 24.0 |
| t_{1/2z} [h] | NC | NC | NC |
| AUC_{0-tz} | 2689 | 8090 | 18548 |
| [ng*h/mL] | | | |

In Vivo Efficacy after Bacterial Infection

In a murine model, infected catheter tubings were implanted under the rodent skin followed by subcutaneous treatment (once per day with 25 mg/kg for six days). This resulted in significant reduction of bacterial load on the catheter fragment and in the tissue surrounding the catheter fragment when compared to the controls. Importantly, subcutaneous treatment with dehalogenil led to >3-log₁₀ reduction in bacterial loads found on the catheter fragments (3.85-log 10) and the tissues (3.24-log 10) surrounding the catheter fragments, respectively, suggesting a high antibacterial activity of dehalogenil on S. aureus in this in vivo infection model.

FIG. 20 shows the in vivo efficacy of dehalogenil in a S. aureus related murine foreign body infection model. The infection was established using a bacterial inoculum of 1×10⁴ colony-forming units (CFU) injected into the lumen of the implanted peripheral venous catheter tubing fragment. Dehalogenil (25 mg/kg) was administered s.c. once per day, starting at 3 h post infection. Linezolid (Zyvoxid, Pfizer) was used as a reference antibiotic. Six days post infection, mice were euthanized, edema sizes around the implanted catheter fragment determined (c), and the catheter fragments and surrounding tissues were explanted. Bacterial loads from catheter detached biofilms (a) and in surrounding tissue homogenates (b) were determined by CFU counting. The data represent the value of every individual animal (symbols) and the median (horizontal lines). *p>0.05, **p<0.01, ***p<0.001 (Kruskal-Wallis tests followed by Dunn's post hoc test).

Dehalogenil was further tested in a Staphylococcus blood infection (sepsis) model, alongside linezolid as reference compound. Mice were infected using S. aureus USA300LAC (10⁸ CFU/animal) and subsequently treated with a daily total of 5, 10 or 25 mg/kg SC dehalogenil, split into two doses (8 h interval, BID regimen) until 3 days post infection. Both 10 and 25 mg/kg resulted in 80 and 90% survival, compared to only 20% in the untreated control, while 60 mg/kg Linezolid resulted in the survival of all animals. It has been demonstrated that dehalogenil is able to rescue severely infected mice, which underlines the potential of this promising class of natural compounds after rational, semisynthetic optimization.

FIG. 21 shows the survival rates in a sepsis model with dehalogenil, vehicle and linezolid (60 mg/kg) given subcutaneously in CD-1 mice (n=10). Mice were infected intravenously using S. aureus USA300LAC (10⁸ CFU/mice). Compounds were administered twice daily starting day 0 until day 3, with the daily total dose indicated. Curves were compared via Mantel-Cox test (Prism), with ***: p<0.001 and **: p<0.01.

Activity Against S. epidermidis Biofilms

Activity of chlorotonils was tested against S. epidermidis RP62A biofilm using two different setups. Initially, a crystal violet assay was performed (FIG. 23, A). Here, a biofilm is formed for 24 h on the bottom of a flat 96-well plate, washed once using PBS prior to addition of challenging medium (MHB2 containing 80×MIC of indicated antibiotic). After 24 h incubation at 37° C., medium was carefully removed and biofilm was stained using 0.1% (w/v) crystal violet solution. After washing with PBS, plates were air-dried and then 33% acetic acid was added to dissolve violet crystals followed by absorbance measurements using a standard plate reader at 600 nm. Chlorotonils were found to prevent biofilm formation as opposed to linezolid or ciprofloxacin. Subsequently, an MBEC (minimal biofilm eradicating concentration) assay was performed using standard MBEC biofilm inoculator plates (Innovotec) (FIG. 23, B). Briefly, biofilm was formed on the pegs for 24 h before being transferred to a challenge plate containing different concentrations dehalogenil. After 24 h incubation, the biofilm was retrieved from the pegs into recovery medium using sonication, diluted several times in PBS before plating on non-selective agar plates to perform a viable cell count. The minimal biofilm inhibiting concentration was found to be 0.5 μg/mL while the MBEC was 4 μg/mL. For the first time, anti-biofilm activity of chlorotonils was confirmed, clearly offering additional opportunities for target product profiles.

1. A compound of general formula (I): 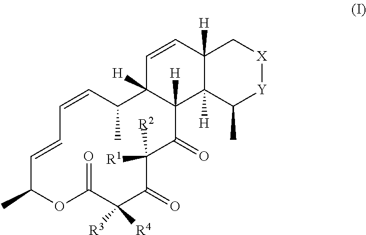
wherein
X—Y together are a group of formula 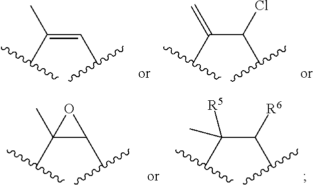
R¹ is hydrogen, F, Cl, S₂H or a methyl group;
R² is hydrogen, F, Cl or a methyl group;
R³ is hydrogen or a methyl group;
R⁴ is hydrogen or a methyl group; and
R⁵ is OH and R⁶ is a halogen atom, OH, ONO₂ or a group of formula —O—C₁₋₆ alkyl which group may be substituted by one or two hydroxy groups and/or by a phenyl group; or
R⁵ is a halogen atom, OH, ONO₂ or a group of formula —O—C₁₋₆ alkyl which group may be substituted by one or two hydroxy groups and/or by a phenyl group and R⁶ is OH;
or a salt thereof;
wherein the following compounds and all tautomeric forms and all salts thereof are excluded:

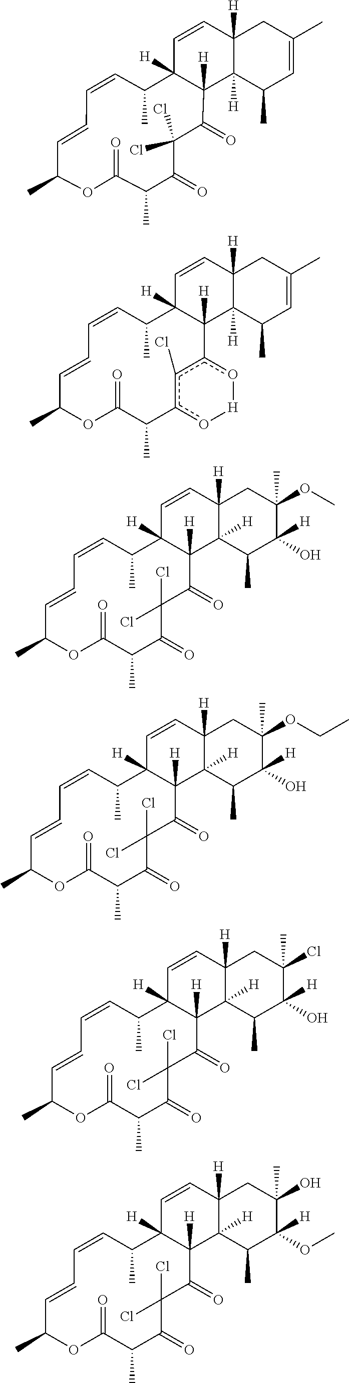

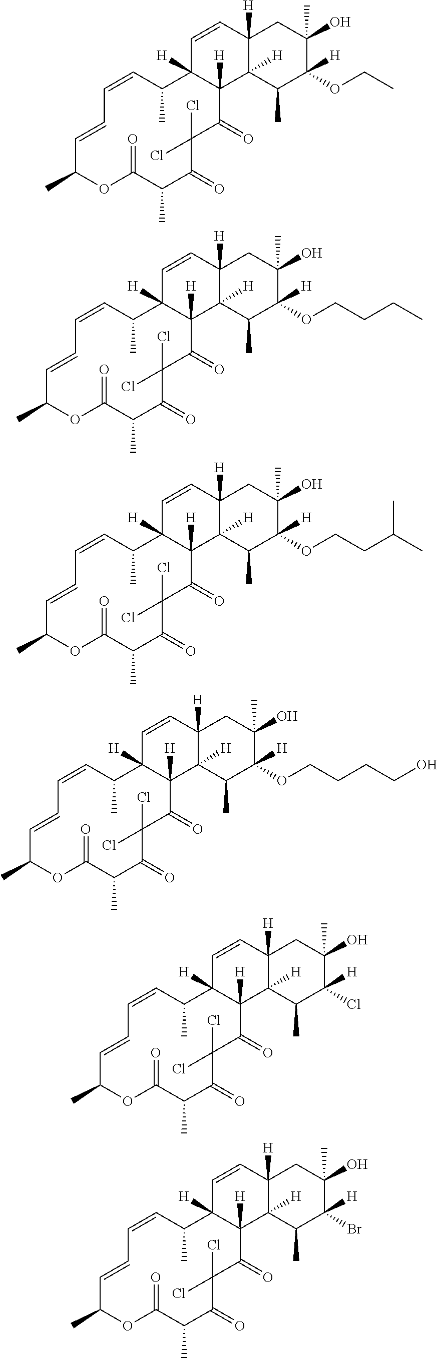

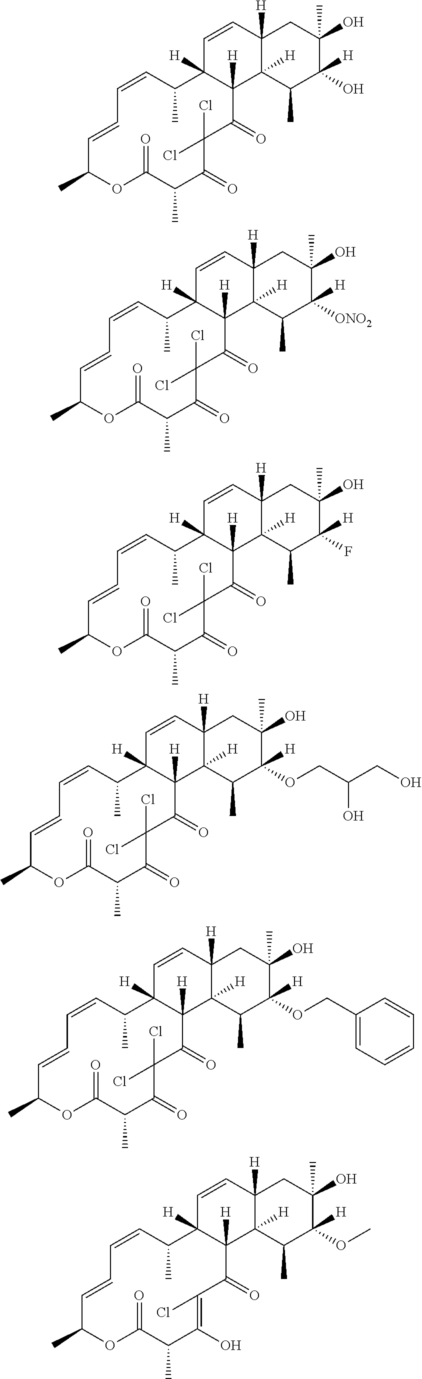

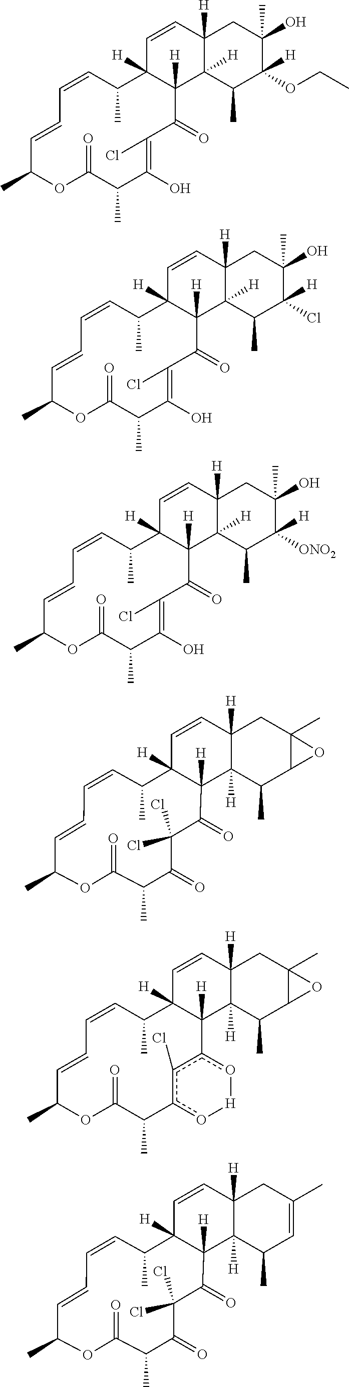

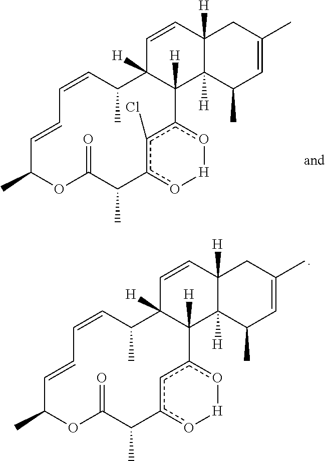

2. A compound according to claim 1 of general formula (II): 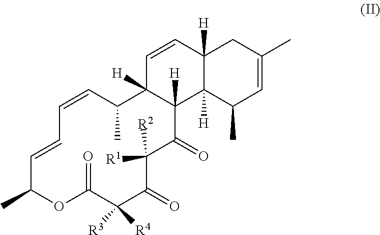
or a salt thereof.

3. A compound according to claim 1 wherein R¹ is hydrogen or Cl.

4. A compound according to claim 1 wherein R¹ is hydrogen.

5. A compound according to claim 1, wherein R² is F or a methyl group.

6. A compound according to claim 1, wherein R² is hydrogen.

7. A compound according to claim 1, wherein R³ is a methyl group.

8. A compound according to claim 1, wherein R⁴ is hydrogen.

9. A compound according to claim 1 having the following structure: 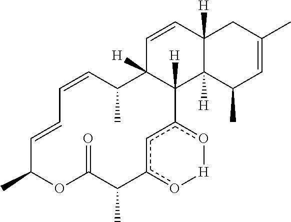
or a salt thereof.

10. A compound according to claim 1 which is selected from the following compounds: 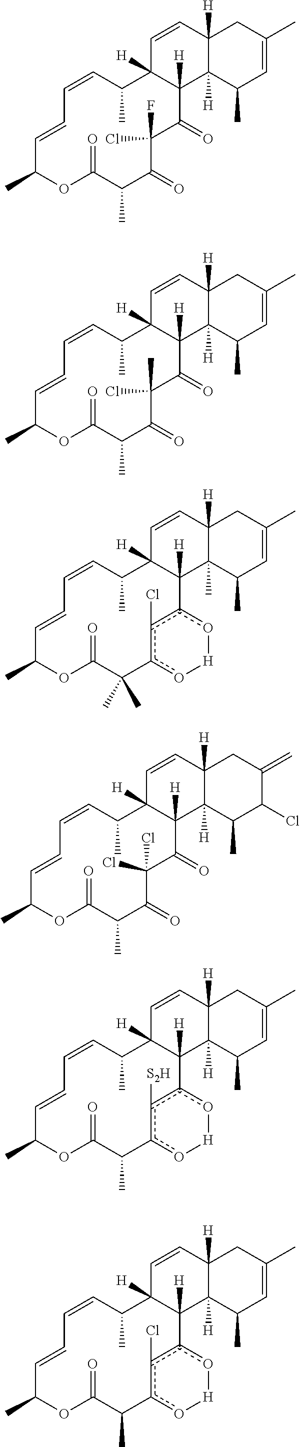 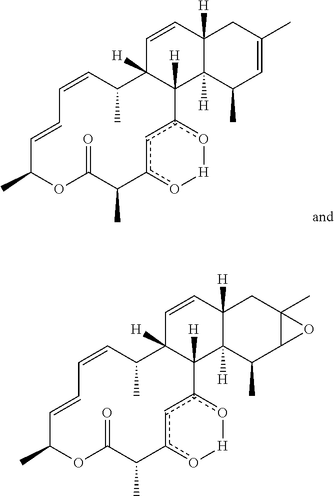
or a salt thereof.

11. Pharmaceutical composition comprising a compound according to claim 1 and one or more carrier substances and/or one or more adjuvants.

12-17. (canceled)

18. A method for the treatment or prophylaxis of a bacterial infection in a subject which comprises administering to the subject an effective amount of a compound according to claim 1.

19. A method for the treatment or prophylaxis of a bacterial infection caused by Staphylococcus spp, and/or Enterococcus spp. in a subject which comprises administering to the subject an effective amount of a compound according to claim 1.

20. A method for the treatment or prophylaxis of malaria in a subject which comprises administering to the subject an effective amount of a compound according to claim 1.

21. A method for the preparation of the following compound: 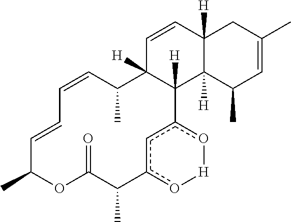
characterized by that chlorotonil B1: 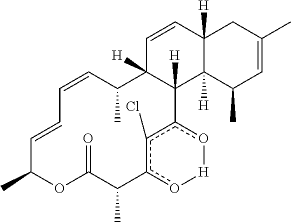
is reacted with sulfur and KOtBu; or
characterized by that chlorotonil A: 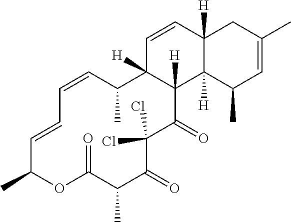
is irradiated by blue LEDs in the presence of (Ir[dF(CF₃)ppy]₂(dtbpy))PF₆, formic acid and DIPEA.

22-23. (canceled)

24. A method for the treatment or prophylaxis of malaria or of a bacterial infection in a subject which comprises administering to the subject an effective amount of a compound selected from the following compounds:

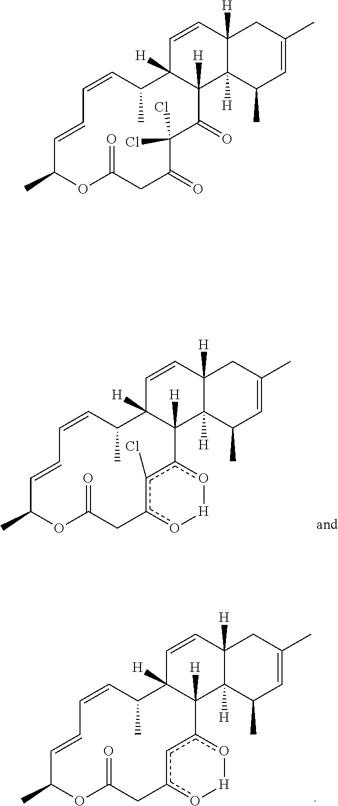

25. (canceled)

26. The method of claim 18 wherein the subject is identified as suffering from a bacterial infection an effective amount of the compound is administered to the identified subject.

27. The method of claim 20 wherein the subject is identified as suffering from malaria an effective amount of the compound is administered to the identified subject.

28. The method of claim 24 wherein the subject is identified as suffering from malaria and an effective amount of the compound is administered to the identified subject.

29. The method of claim 24 wherein the subject is identified as suffering from a bacterial infection an effective amount of the compound is administered to the identified subject.
